(19) United States
(12) Patent Application Publication
(10) Pub. No.: US 2013/0273049 A1
(43) Pub. Date: Oct. 17, 2013

(54) ROBO1-FC FUSION PROTEIN FOR USE IN THE TREATMENT OF HEPATOCARCINOMA

(71) Applicants: Frédérique Dol-Gleizes, Paris (FR); Geneviève Gueguen-Dorbes, Paris (FR)

(72) Inventors: Frédérique Dol-Gleizes, Paris (FR); Geneviève Gueguen-Dorbes, Paris (FR)

(73) Assignee: SANOFI, Paris (FR)

(21) Appl. No.: 13/996,689

(22) PCT Filed: Dec. 22, 2011
(86) PCT No.: PCT/EP2011/073739

§ 371 Date: Jun. 21, 2013

(30) Foreign Application Priority Data
Dec. 23, 2010 (FR) 1061163
Oct. 14, 2011 (EP) 11306336.6

(51) Int. Cl. C07K 16/28

(52) U.S. Cl. CPC C07K 16/28
USPC 424/134.1

(57) ABSTRACT

The present invention relates to the use a Robo1-Fc recombinant protein for treating cancer, in particular hepatocarcinoma.

The present invention relates to a recombinant protein Robo1-Fc and to the use thereof for treating diseases in which a Slit protein is overexpressed. In particular, the present invention relates to the use of a Robo-Fc protein for treating cancer, especially hepatocarcinomas. It also relates to a composition comprising such a recombinant protein. Another aspect of the invention consists of the use of a Robo1-Fc molecule as a diagnostic tool for detecting the overexpression of a molecule of the Slit family in a patient.

Slit ligands were first of all described for their role in repelling axonal growth in neuronal development. Since then, the Robo/Slit regulatory pathway has also been described in tumour angiogenesis. Specifically, the Robo4/Slit2 pathway has been described for inhibiting the response of endothelial cells to VEGF (Jones C. A. et al. Nat. Med 14, 448-453 (2008)). Regulation by the Robo/Slit pathway makes it possible to channel the excessive proliferation of endothelial non-mature neovessels or buds (non-productive angiogenesis) and to mature these vessels. The expression of Slit2 at the transcriptional level has been demonstrated in several human cancer lines, in particular HCT116 derived from colon carcinoma, Skov-3 derived from ovarian carcinoma, HeLa derived from cervical cancer, MDA-MB-435 derived from melanoma, Hec-1A derived from uterine cancer and, finally, 769-P derived from renal carcinoma (Stella M C et al., Mol Biol Cell. 2009 Vol. 20, Issue 2, 642-657). Overexpression of the Slit2 protein has also been demonstrated on human tissues derived from carcinomas: oral carcinoma (Wang et al. 2008, Cancer Sci. 2008 March; 99(3): 510-7), prostate carcinoma (Latil et al. 2003 Int J Cancer. 2003 Jan. 20; 103(3): 306-15), colon carcinoma (Wang et al. 2003, Cancer Cell, Volume 4, Issue 1, July 2003, Pages 19-29), and liver carcinoma (Avci et al, 2008, BMC cancer, 8: 392). More recently, overexpression of the Slit2 protein has been shown on samples of endometriosis (Shen et al, 2009, AJP 175 (2): 479).

The Robo1 protein exists as two isoforms a and b. The extracellar domain of the Robo1 protein isoform b (NP_{—}598334) comprises 5 immunoglobulin domains: Ig1, Ig2, Ig3, Ig4 and Ig5. The Robo protein interacts with the Slit ligands at the level of the Ig1 and Ig2 domains. Liu et al. (2004, Mol. Cell Neurosci, 26: 232-240) have demonstrated the importance of the Ig2 domain in the interaction with Slit and in the activity of Robo (chemorepulsion).

The use of antibodies specific for the Robo1 and the Slit2 proteins has been described in application WO2003/075860. These antibodies make it possible to inhibit tumour angiogenesis.

However, it would be advantageous to propose an alternative approach for treating cancer, in particular a molecule capable of inhibiting the Slit2 signalling pathway.

The inventors have developed a strategy of soluble chimeric Robo1 receptors capable of binding Slit ligands and consequently, of inhibiting the intracellular signalling of the Robo/Slit pathway. Surprisingly, the Robo1-Fc molecules according to the invention have an anti-angiogenesis effect by preventing the formation of mature vessels and not by inhibiting the proliferation of endothelial cells.

The inventors have shown that the Robo1-Fc molecules also have an antitumour effect in lung and liver cancer.

### DETAILED DESCRIPTION OF THE INVENTION

A subject of the present invention is a Robo-Fc recombinant protein comprising the extracellular domain of the Robo1 protein, isoform b or a part of this domain, a junction region (linker) and an immunoglobulin Fc domain.

In one particular embodiment, the extracellular domain of the Robo1 protein, isoform b consists of the Ig1 and Ig2 domains. These domains correspond to the peptide of SEQ ID NO.2 encoded by the nucleotide sequence SEQ ID NO.1, or a sequence having at least 80%, 85%, 90%, 95% or 99% identity with the sequence SEQ ID NO.2.

The fusion protein also comprises a junction region, also called “linker”. In the context of the present invention, the linker makes it possible to give the recombinant protein stability, in particular by limiting in vivo cleavage. Linkers that can be used in a Robo1-Fc molecule are, for example, GluArgProSerPheVal and GlyGlyGlyGlySer. Those skilled in the art have sufficient knowledge to select a linker suitable for this use.

The Fc domain of the Robo1-Fc recombinant molecules according to the invention corresponds to a crystallizable fragment of an immunoglobulin. This Fc fragment can come from various immunoglobulins IgG1, IgG2, IgG3 or IgG4. It is responsible for the effector function of the immune response (WO2008/065543).

In one embodiment according to the invention, the Fc domain comes from an IgG4 immunoglobulin. In one particular embodiment, at least one point mutation or deletion has been introduced into the IgG4 Fc domain so as to increase the stability of the molecule, in particular by stabilizing the hinge region made up of the two Fc domains (Angla et al., 1993, Mol. Immunol., 30: 105-108), to reduce or eliminate the residual activity of the IgG4-Fc, in particular the effector activity (WO 97/09351), and to increase the homogeneity during the production of the recombinant protein. In particular, at least two point mutations, preferably three point mutations, have been introduced into the IgG4 Fc domain. In the Robo1-Fc L1, Robo1-Fc L2 and Robo1-Fc L3 molecules according to the invention, the preferred mutations are the following:
S241P (Kabat numbering) in order to stabilize the disulphide-bridge bonding in the Fc hinge region;
L248E (Kabat numbering) in order to eliminate the residual effector activity of the IgG4-Fc domain;
absence of the C-terminal lysine in order to reduce the heterogeneity of the protein.

Robo1-Fc molecules according to the invention comprising the Ig1 and Ig2 domains of Robo1 isoform b, a linker and a mutated IgG4 domain as described above are Robo1-Fc L1, Robo1-Fc L2 and Robo1-Fc L3.

Robo1-Fc L1 corresponds to the protein of sequence SEQ ID NO.4, encoded by the nucleotide sequence SEQ ID NO.3.

Robo1-Fc L2 corresponds to the protein of sequence SEQ ID NO.6, encoded by the nucleotide sequence SEQ ID NO.5.

Robo1-Fc L1 and Robo1-Fc L2 differ by virtue of the nature of the linker.

Robo1-Fc L3 corresponds to the protein of sequence SEQ ID NO.24, encoded by the nucleotide sequence SEQ ID NO.23.

In one particular embodiment of the invention, one or more point mutations or deletions have been introduced in order to increase the homogeneity during production. In particular, one amino acid, preferably two amino acids, have been truncated in the N-terminal position. Such a Robo-1-Fc molecule according to the invention is the Robo1-Fc L3 molecule, in which the two amino acids (Ser20 and Arg21) have been truncated and in which the amino acid Leu 22 has been fused to the signal peptide of interleukin 2.

The homogeneity of the Robo1-Fc molecules according to the invention is also increased by deleting the C-terminal Lys as previously described.

A subject of the invention is also proteins of which the protein sequence corresponds to the sequences SEQ ID NO.2, SEQ ID NO.4, SEQ ID NO.6 or SEQ ID NO.24 or has at least 80%, 85%, 90%, 95% or 99% identity with the sequences SEQ ID NO.2, SEQ ID NO.4, SEQ ID NO.6 or SEQ ID NO.24. These protein variants have the same biological activity as the proteins having the sequence SEQ ID NO.2, SEQ ID NO.4, SEQ ID NO.6 or SEQ ID NO.24, in particular their ability to interact with the proteins of the Slit family.

The Robo1-Fc proteins according to the invention can be produced by transfection of expression plasmids encoding these proteins into any cell type suitable for the expression of eukaryotic recombinant proteins: HEK293, CHO, etc., and then recovered in the culture supernatant and purified according to conventional methods.

Characterization of the Robo1-Fc L1, Robo1-Fc L2 and Robo1-Fc L3 proteins according to the invention has made it possible to confirm that they have the qualities required for their administration as a biotherapeutic agent.

A Robo1-Fc protein according to the invention has the ability to interact with a protein of the Slit family.

The Robo1-Fc proteins according to the invention specifically recognize the human Slit1, Slit2 and Slit3 proteins and the murine Slit2 protein, in particular by interaction with their D2 domain. Their affinity is similar with respect to the 3 members of the Slit family.

Another subject according to the invention corresponds to the nucleic acid molecules encoding the proteins according to the invention.

Thus, the nucleic acid molecules corresponding to the sequences SEQ ID NO.1, SEQ ID NO.3, SEQ ID NO.5 or SEQ ID NO.23 or having at least 80%, 85%, 90%, 95% or 99% identity with the molecules having the sequence SEQ ID NO.1, SEQ ID NO.3, SEQ ID NO.5 or SEQ ID NO.23 are part of the invention.

Another subject according to the invention consists of the use of a Robo1-Fc protein according to the invention for treating diseases in which a protein of the Slit family is overexpressed.

The proteins of the Slit family which can be targeted by the Robo1-Fcs according to the invention are Slit1, Slit2 or Slit3.

It has been shown that Robo1 can interact with the various members of the Slit family. Consequently, it is advantageous to note that the Robo1-Fc proteins according to the invention are capable of simultaneously inhibiting the signalling pathways mediated by Slit1, Slit2 and Slit3, which makes it possible to broaden the therapeutic spectrum in comparison with the antibodies which are specific for only one of these pathways.

In another embodiment, a Robo1-Fc protein is used for treating diseases in which a protein of the Slit family is overexpressed, by inhibiting angiogenesis without inhibiting endothelial cell proliferation. This anti-angiogenic activity linked to a vessel maturation defect is called “non-productive angiogenesis”.

Specifically, the experimental studies have shown that the Robo1-Fc molecules according to the invention are capable of inhibiting the formation of tubules, without inhibiting endothelial cell proliferation. They make it possible to very significantly reduce the tumour volume in a murine model of lung cancer.

In one preferred embodiment, the Robo1-Fc proteins that can be used for treating diseases in which a Slit protein is overexpressed are Robo1-Fc L1, Robo1-Fc L2 and Robo1-Fc L3, and the molecules which are derived therefrom, include, in particular, point mutations or deletions aimed at increasing the homogeneity during production without significantly modifying the biological properties of these molecules.

In general, the diseases that can be treated with a Robo-1-Fc protein according to the invention are all the diseases in which inhibition of the Slit pathway can have a therapeutic effect, in particular the diseases in which a protein of the Slit family is overexpressed, and in particular those in which Slit2 is overexpressed.

Since the Robo1-Fc molecules according to the invention specifically bind the Slit2 molecule, it is interesting to note that said molecules are capable of simultaneously inhibiting the two pathways in which Slit2 is involved, namely the Robo1/Slit2 and Robo4/Slit2 pathways.

In a specific embodiment of the invention, the Robo1-Fc proteins according to the invention is used for treating cancer, in particular pancreatic cancer, colon cancer, colorectal cancer with or without lymphatic metastasis, breast cancer, lung cancer and lung metastases, ovarian cancer, cervical cancer, melanomas, renal cancer, oral cancer, prostate cancer, liver cancer, etc. In a specific embodiment, said cancer is a cancer in which Slit2 is overexpressed.

In one particular embodiment, the Robo-Fc proteins are used for treating lung cancer.

In another particular embodiment, the Robo-Fc proteins are used for treating liver cancer, such as e.g. hepatocarcinoma, hepatocellular carcinoma (HCC), fibrolamellar carcinoma (a pathological variant of hepatocellular carcinoma), cholangiocarcinoma, hepatoblastoma, or angiosarcoma of the liver.

The Robo1-Fc molecules according to the invention can also be used as an anticancer medicament as an alternative to or as a supplement to the current therapies.

In particular, these molecules can be administered in combination (optionally simultaneously) with anticancer compounds.

Another subject of the invention relates to a composition comprising a Robo1-Fc protein as defined above and one or more pharmaceutically acceptable excipients.

The Robo1-Fc proteins according to the invention can be formulated in pharmaceutical compositions with a view to topical, oral, parenteral, intranasal, intravenous, intramuscular, subcutaneous, intraocular, etc., administration. Preferably, the pharmaceutical compositions contain pharmaceutically acceptable vehicles for an injectable formulation. They may in particular be isotonic sterile saline solutions (monosodium or disodium phosphate, sodium, potassium, calcium or magnesium chloride, etc., or mixtures of such salts), or dry, in particular freeze-dried, compositions which, by addition, as appropriate, of sterilized water or of physiological saline, allow the formation of injectable solutes.

Another aspect of the invention consists of the use of a Robo1-Fc molecule as a diagnostic tool for detecting the overexpression of a molecule of the Slit family in a patient. This is because it has been shown that the Slit pathway is implicated in many cancers. The provision of a test for evaluating a disturbance of the Slit signalling pathway is very useful for the purpose of selecting patients capable of responding to a treatment based on the administration of a Robo1-Fc molecule.

This diagnostic tool may be in the form of a ready-to-use kit, comprising a Robo1-Fc molecule in a form suitable for it to be brought into contact with a biological sample from a patient (blood, urine, tumour biopsy) liable to exhibit an overexpression of a Slit molecule. The Robo-Fc molecule can optionally be prelabelled, and the combination of Robo-Fc and Slit is detected so as to evaluate an increase in the expression of a Slit protein in the biological sample in comparison with a control sample. This kit can, for example, be in the form of an ELISA kit.

### DESCRIPTION OF THE FIGURES

FIG. 1: Expression plasmids for the Robo1-Fc L1, Robo1-Fc L2 and Robo1-Fc L3 proteins.

FIG. 2: Evaluation of the interaction of the Robo1-Fc fusion protein variants with the Slit2 protein by ELISA.

FIG. 3: Affinity of Robo1-Fc for HEK293 cells not expressing Slit2, measured by FACS.

FIG. 4: Affinity of Robo1-Fc for HEK293 cells expressing Slit2, measured by FACS.

FIG. 5: Pharmacokinetic profile of the Robo1-Fc proteins after an iv injection in mice. A. Administration of Robo1-Fc L1, B. Administration of Robo1-Fc L2.

FIG. 6: Effect of the Robo1-Fc L1 molecule in a coculture test with endothelial cells and mesenchymal cells. A. Robo1-Fc L1 inhibits tubule formation in culture. B. Robo1-Fc L1 significantly inhibits VEGF-induced pseudotubule formation.

FIG. 7: Evaluation of the effect of the Robo1-Fc L1 molecule on an ex vivo aortic ring model in mice. A: Description of the protocol for preparing an aortic ring and for measuring tubules. B: a. Control; b. Robo1-Fc Slit2-minus 500 μg/mL+VEGF 10 ng/mL; c. Robo1-Fc L1 500 μg/mL+VEGF 10 ng/mL.

FIG. 8: Mean tumour weight (HepG2 cells in the left lobe of the liver of SCID mice) 28 days after injection of the cells, in the control group and in the group having received 4 injections of Robo1-Fc at 25 mg/kg.

FIG. 9: Mean tumour weight (Hep3B cells one lobe of the liver of SCID mice) 28 days after injection of the cells, in the control group and in the group having received 5 mg/kg of Robo1-Fc twice a week.

FIG. 10: Plasmatic AFP concentration in the control group and in the group having received 5 mg/kg of Robo1-Fc twice a week.

### EXAMPLES

### Example 1

### Preparation of the Robo1-Fc proteins

a. Constructs Allowing the Expression of the Robo1-Fc Recombinant Proteins Used as Biotherapeutic Agents.

The Robo1-Fc recombinant proteins consist of a fusion between the first two immunoglobulin domains (Ig1-Ig2) of the human Robo1 protein, isoform b (NP_{—}598334) and the Fc domain of human immunoglobulin G4 (hIgG4-Fc, SwissProt IGHG4_HUMAN).

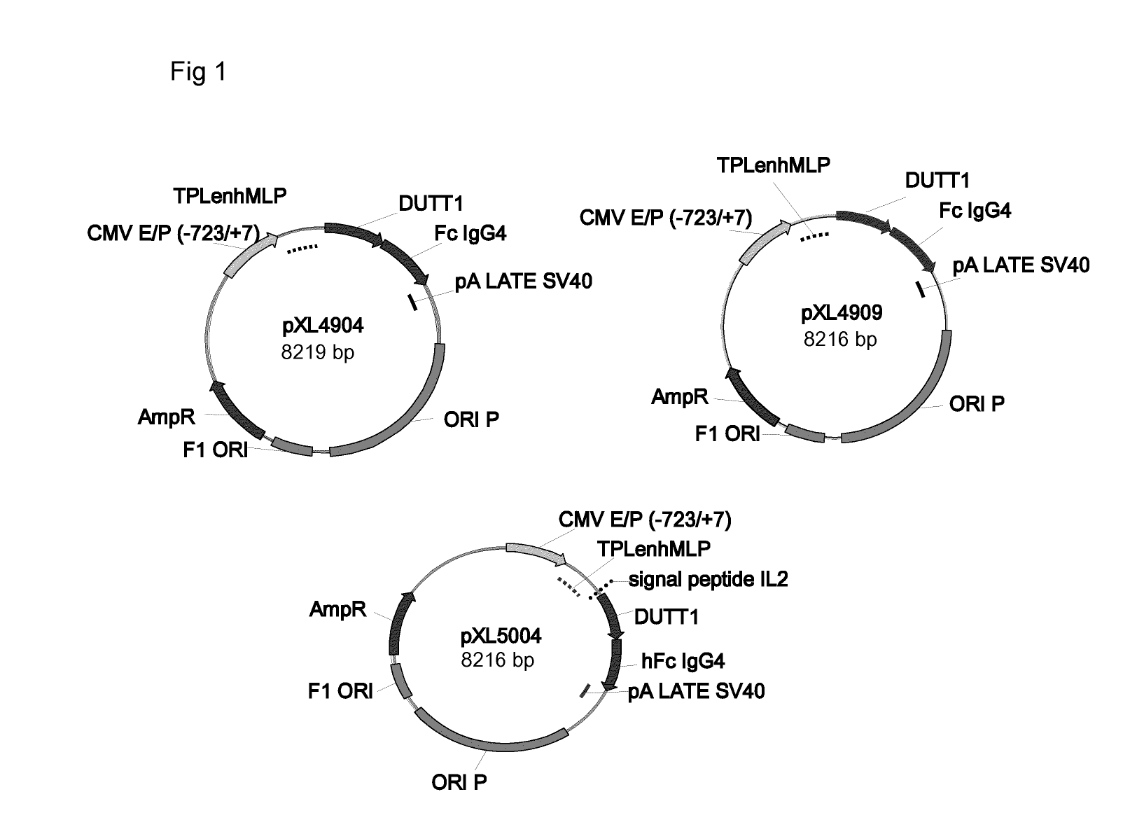

In order to obtain the Robo1-Fc L1 construct, a fragment of the cDNA (SEQ ID NO.1) corresponding to the immunoglobulin (Ig) domains Ig1 and Ig2 of this protein (SEQ ID NO.2) followed by a GluArgProSerPheVal linker was amplified by PCR using the human foetal heart cDNA library (ref. HL5042T, Clontech). This amplified fragment was then cloned into the eukaryotic expression vector pXL4904 (described in FIG. 1) such that the two Ig domains of Robo1 are expressed as a fusion with the Fc domain of human IgG4 in the C-terminal position. Three point mutations were introduced into the IgG4-Fc domain in order to obtain the following characteristics: S241P (Kabat numbering) in order to stabilize the disulphide-bridge bonding in the Fc hinge region; L248E in order to eliminate the residual effector activity of the IgG4-Fc domain; absence of the C-terminal lysine in order to reduce the heterogeneity of the protein. The cDNA sequence used to express this recombinant protein corresponds to the sequence SEQ ID NO.3. The recombinant protein obtained is called Robo1-Fc L1 and corresponds to the protein sequence SEQ ID NO.4.

In order to obtain the Robo1-Fc L2 construct, the same cDNA corresponding to the Ig1-Ig2 domains but without the linker mentioned was cloned into the eukaryotic expression vector pXL4909 (described in FIG. 1), which allows the expression of these Ig domains as a fusion with the same Fc domain of IgG4 containing the 3 point mutations described in the construction of Robo1-Fc L1, but this time introducing a GlyGlyGlyGlySer linker upstream of the Fc domain. The cDNA sequence used to express this recombinant protein corresponds to the sequence SEQ ID NO.5. The recombinant protein obtained is called Robo1-Fc L2 and corresponds to the protein sequence SEQ ID NO.6.

b. Construction of the Robo1-Fc Proteins Used as Controls

In order to obtain the Robo1-Fc Slit2-minus construct, the cDNA previously cloned into the pXL4904 plasmid was modified by PCR so as to introduce the point mutations allowing the substitutions of the leucine at position 38 to glutamine and the phenylalanine at position 89 to tyrosine. The cDNA sequence used to express this recombinant protein corresponds to the sequence SEQ ID NO.7. The recombinant protein obtained is called Robo1-Fc Slit2-minus and corresponds to the protein sequence SEQ ID NO.8.

In order to obtain the Robo1-Fc heparin-minus construct, the cDNA cloned into the pXL4904 plasmid was modified by PCR so as to introduce the point mutations allowing the substitutions of the arginine at position 97 to aniline and the lysine at position 98 to alanine. The cDNA sequence used to express this recombinant protein corresponds to the sequence SEQ ID NO.9. The recombinant protein obtained is called Robo1-Fc heparin-minus and corresponds to the protein sequence SEQ ID NO. 10.

c. Production of the Various Robo1-Fc Proteins

The two proteins Robo1-Fc L1 and Robo1-Fc L2 were produced by transient transfection in the HEK293 line (FreeStyle 293-F cells ref 51-0029, Invitrogen, according to the supplier's recommendations) using the pXL4904 and pXL4909 plasmids, respectively, and the helper plasmids pXL4544 and pXL4551 allowing the expression of two N-glycan glycosylation enzymes, i.e. α-2,3-sialyltransferase and β-1,4-galactosyltransferase, as has been described in application WO2008/065543. These proteins were also produced by transfection in the CHO line (CHO-S cells, ref 11619-012, Invitrogen, according to the supplier's recommendations) using the pXL4904 and pXL4909 plasmids, respectively.

The Robo1-Fc L1 and Robo1-Fc L2 proteins expressed in the culture natant of the HEK293 cells were purified by chromatography on a protein A affinity column (MabSelect ref. 17-5199-02, Amersham Biosciences) and elution in 20 mM NaCl/100 mM acetic acid buffer, and then formulated in PBS buffer (ref. 14190-094, Invitrogen).

The Robo1-Fc Slit2-minus and Robo1-Fc heparin-minus recombinant proteins were produced and purified in the same way.

d. Physicochemical Characterization of the Robo1-Fc Recombinant Proteins

SDS-PAGE and gel permeation analysis made it possible to show that the proteins were dimeric and more than 96% pure. Mass spectrometry analysis made it possible to demonstrate the identity of these proteins, the measured weight of the deglycosylated protein being in perfect agreement with the weight calculated in silico. Analysis of the monosaccharide composition and quantification of the N-glycan sialic acids as described by Saddic et al. 2002. (Methods Mol. Biol. 194: 23-36 and Anumula et al. 1998. Glycobiology 8: 685-694) made it possible to demonstrate that the proteins were sialylated to a very great extent. The results are given in table 1. It will be noted that the N-terminal analysis of the Robo1-Fc L1 and Robo1-Fc L2 molecules showed that these purified proteins contained a variable proportion (0 to 40%) of a form with the first 2 residues (Ser20 and Arg21) truncated.

TABLE 1
Robo1-Fc protein identity

| | Robo1-Fc L1 | | Robo1-Fc L2 | |
|---|---|---|---|---|
| Weight (SDS-PAGE | 56 | kDa | 55 | kDa |
| reducing conditions) | | | | |
| Weight (SEC, HPLC) | 180 | kDa | 180 | kDa |
| Weight (LC/MS after | 48812 | Da | 48410 | Da |
| deglycosylation) | | | | |

| | | |
|---|---|---|
| N-Glycan profile | highly sialylated | highly sialylated |
| (HPLC) | 21.4% asialoglycans | 20.1% asialoglycans |
| Monosaccharide | complex N-glycans | complex N-glycans |
| composition (HPLC) | entirely fucosylated | entirely fucosylated |
| | no O-glycans | no O-glycans |

### Example 2

### Preparation of the Slit Proteins Used as Ligand

The cDNA encoding the human Slit2 protein corresponds to the reference protein NP_{—}004778. Fragments of this cDNA were amplified by PCR using the human brain cDNA library (ref. 639300, Clontech).

The cDNA corresponding to the D2 domain was cloned into the eurkaryotic expression vector pXL4911 in order to express this domain containing a His tag in the C-terminal position. The cDNA sequence used to express this recombinant protein corresponds to the sequence SEQ ID NO.11. The recombinant protein obtained is called Slit2-D2 and corresponds to the protein sequence SEQ ID NO.12.

The cDNA corresponding to the D1-D2 domains was cloned into the eukaryotic expression vector pXL4912 in order to express these two domains containing a His tag in the C-terminal position. The cDNA sequence used to express this recombinant protein corresponds to the sequence SEQ ID NO.13. The recombinant protein obtained is called Slit2-D1D2 and corresponds to the protein sequence SEQ ID NO.14.

The cDNA corresponding to the extracellular part (Nt) of the Slit2 protein was cloned into the eukaryotic expression vector pXL5033 in order to express this protein with a His tag in the C-terminal position. The cDNA sequence used to express this recombinant protein corresponds to the sequence SEQ ID NO.15. The recombinant protein obtained is called Slit2-N and corresponds to the protein sequence SEQ ID NO.16.

The cDNA encoding the D2 domain of the murine Slit2 protein, and corresponding to the D2 domain of the described reference protein NP_{—}848919, was obtained from the cDNA cloned into the pXL4911 plasmid. Four fragments making it possible to generate the five point mutations were generated by PCR with pXL4911 as template, and then these fragments were used as template to amplify the cDNA encoding the complete D2 domain by sequential PCR. The protein carries the Thr311Ser, Lys313Arg, Ile329Leu, Ile411Val and Pro418Ala mutations allowing it to be identical to the D2 domain of the murine Slit2 protein. This plasmid makes it possible to express the D2 domain of the murine Slit2 protein with a His tag in the C-terminal position. The cDNA sequence used to express this recombinant protein corresponds to the sequence SEQ ID NO.17. The recombinant protein obtained is called mSlit2-D2 and corresponds to the protein sequence SEQ ID NO.18.

The cDNA encoding the human Slit1 protein corresponds to the described reference protein NP_{—}003052. A fragment of this cDNA was amplified by PCR using the human brain cDNA library (ref. 639300, Clontech). The cDNA corresponding to the D2 domain was cloned into the eukaryotic expression vector pXL5020 in order to express this domain containing a His tag in the C-terminal position. The cDNA sequence used to express this recombinant protein corresponds to the sequence SEQ ID NO.19. The recombinant protein obtained is called Slit1-D2 and corresponds to the protein sequence SEQ ID NO.20.

The cDNA encoding the human Slit3 protein corresponds to the described reference protein NP_{—}003053. A fragment of this cDNA was amplified by PCR using the human brain cDNA library (ref. 639300, Clontech). The cDNA corresponding to the D2 domain was cloned into the eukaryotic expression vector pXL5021 in order to express this domain containing a His tag in the C-terminal position. The cDNA sequence used to express this recombinant protein corresponds to the sequence SEQ ID NO.21. The recombinant protein obtained is called Slit3-D2 and corresponds to the protein sequence SEQ ID NO.22.

The three proteins called Slit2-D2, Slit2-D1D2 and Slit2-N were produced by transient transfection in the HEK293 line (FreeStyle 293-F cells according to the supplier's recommendations, Invitrogen) using the pXL4911 plasmid (respectively, pXL4912 or pXL5033).

The Slit2-D2 and Slit2-D1D2 proteins expressed in the culture supernatant of the HEK293 cells were purified by chromatography on an Ni-chelating sepharose column (ref. 17-0409-03, Amersham Biosciences) and elution in imidazole buffer and then formulated in PBS buffer (ref. 14190-094, Invitrogen) adjusted to 1M NaCI. Mass spectrometry analysis (LC/MS) made it possible to demonstrate the identity of these proteins.

The three proteins called mSlit2-D2, Slit1-D2 and Slit3-D2 were produced, purified and characterized in a comparable manner.

### Example 3

### Study of the Affinity of the Robo1-Fc Recombinant Proteins for the Slit Proteins and for Heparin by Means of Three Methods: ELISA, SPR and FACS

a. Affinity of the Robo1-Fc Proteins for Heparin

In order to determine the affinity of the Robo1-Fc constructs for heparin, 2 mg of Robo1-Fc protein, purified and formulated in 10 mM phosphate, pH 7.0, were chromatographed on a heparin column (1 mL HiTrap Heparin-Sepharose HP, GE Heathcare) by elution with a linear gradient of NaCI of from 0 to 1.5 M.

Table 2 indicates the NaCI concentration of 448 mM necessary for eluting the Robo1-Fc L1 protein as described in the literature (Fukuhara, N. et al. 2008 J. Biol. Chem. 283 p 16226-16234).

TABLE 2
Affinity of Robo1-Fc for heparin

| | Detachment from the heparin |
|---|---|
| Robo1-Fc construct | column [NaCl], mM |
| Robo1-Fc L1 | 448 |
| Robo1-Fc Heparin-minus | No binding |

These results show that the Robo1-Fc heparin-minus protein is not retained on this column, and therefore that it no longer has any affinity for heparin. This protein is therefore a heparin-negative mutant.

b. Evaluation of the Interaction of the Robo1-Fc Protein Variants with the D2 Domain of the Human Slit2 Protein

This example describes the interaction of the two variants called Robo1-Fc L1 and Robo1-Fc L2 with their natural ligand (in these experiments, the D2 domain of human Slit2) by ELISA assay.

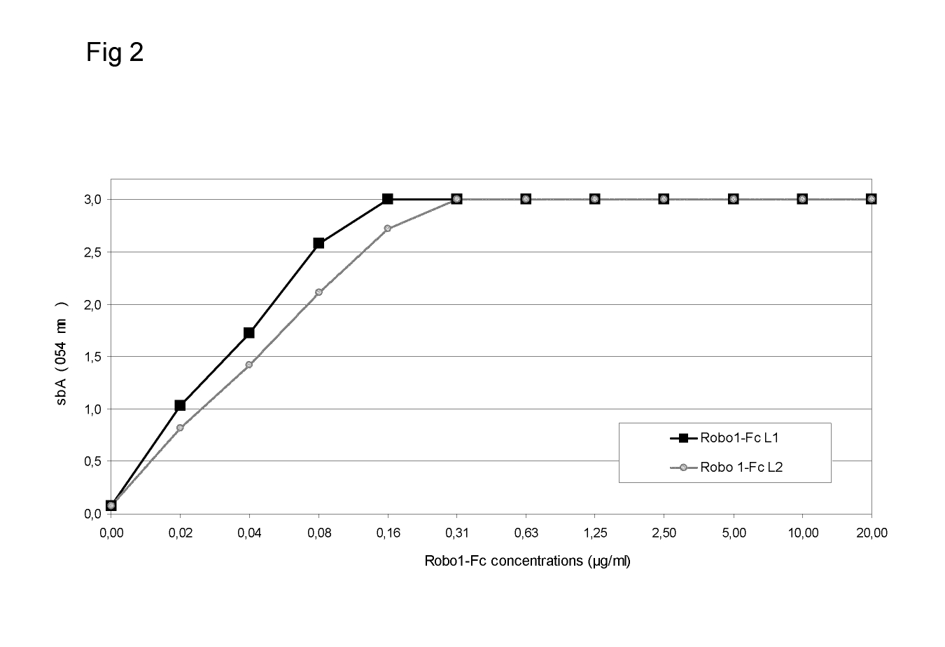

The human Slit2-D2 protein was bound to Immulon-4 enzyme-linked plates (VWR Scientific Inc. Swedesboro, N.J.). A concentration range (from 20 μg/mL to 0.02 μg/mL) of the Robo1-Fc L1 and Robo1-Fc L2 variants was added and then detected by means of the peroxidase-conjugated goat anti-human IgG antibody (Sigma; ref. A0170, dilution to 1:50 000). Visualization was carried out with the TMB-H₂O₂ substrate (Interchim; ref UP664780) and the measurements were carried out with a plate reader at 450 nm. The results are reported in FIG. 2. They show that the two variants Robo1-Fc L1 and L2 specifically interact with the human Slit2 protein (in particular with the D2 domain).

c. Evaluation of the Interaction of the Robo1-Fc Protein with the Human Variants of the Slit Family, Namely Slit1 and Slit3

This example describes the interaction of the Robo1-Fc L1 fusion protein with the Slit1-D2, Slit2-D2 and Slit3-D2 variants by ELISA assay.

The D2 domain of the human Slit variants was bound to Immulon-4 enzyme-linked plates (VWR Scientific Inc. Swedesboro, N.J.). A concentration range (from 1 μg/mL to 0.001 μg/mL) of the Robo1-Fc L1 fusion protein was added and then detected using the peroxidase-conjugated goat anti-human IgG antibody (Sigma; ref. A0170, dilution to 1:50 000). Visualization was carried out with the TMB-H₂O₂ substrate (Interchim; ref UP664780) and the measurements were carried out with a plate reader at 450 nm. Similarly, the Robo1-Fc Slit2-minus variant, which is mutated at the level of the Slit2-binding site, was evaluated according to a concentration range (from 20 μg/mL to 0.02 μg/mL) by ELISA assay under the same conditions described above. The results are reported in table 3 below.

TABLE 3
Affinity of Robo1-Fc for the human variants of the Slit protein

| Slit protein (ligand) | Robo1-Fc protein | EC50 (μg/mL) | CV (%) |
|---|---|---|---|
| Slit2-D2 | Robo1-Fc L1 | 1.32E−01 | 3.3 |
| Slit1-D2 | Robo1-Fc L1 | 7.88E−02 | 3.9 |
| Slit3-D2 | Robo1-Fc L1 | 2.60E−01 | 12 |
| Slit2-D2 | Robo1-Fc Slit2-minus | 1.78E+01 | 18 |

These results show that the Robo1-Fc protein interacts specifically with the three proteins of the family, Slit1, Slit2 and Slit3 (in particular with their D2 domain).

In addition, the Robo1-Fc Slit2-minus protein no longer has affinity for heparin and it is therefore a heparin-negative mutant.

d. Evaluation of the Interaction of the Robo1-Fc Protein with the Murine Slit2 Protein

This example describes the interaction of the Robo1-Fc L1 fusion protein with the murine protein mSlit2-D2 by ELISA assay.

The murine protein mSlit2-D2 was bound to an Immulon-4 enzyme-linked plate (VWR Scientific Inc. Swedesboro, N.J.). A concentration range (from 2 μg/mL to 0.002 μg/mL) of the Robo1-Fc L1 fusion protein was added and then detected using the peroxidase-conjugated goat anti-human IgG antibody (Sigma; ref. A0170, dilution to 1:50 000). Visualization was carried out with the TMB-H₂O₂ substrate (Interchim; ref UP664780) and the measurements were carried out with a plate reader at 450 nm. The results are reported in table 4 below.

TABLE 4
Affinity of Robo1-Fc L1 for the murine Slit2 protein

| Protein studied | EC50 (μg/mL) | CV (%) |
|---|---|---|
| mSlit2-D2 | 2.21E−01 | 18 |

These results show that the Robo1-Fc protein interacts specifically with the murine Slit2 protein

e. Affinity of Robo1-Fc for the Slit Protein Measured by SPR

This example describes the determination of the affinity constant of the Robo1-Fc L1 fusion protein with the human Slit2 protein (in this experiment, Slit2-D2) by SPR (Surface Plasmon Resonance; BIAcore 2000). The interaction between the Robo1-Fc protein and the human Slit2 protein was analysed after having bound the Robo1-Fc fusion protein to a CM5 chip. The kinetic measurements were carried out according to the protocol of Canziani et al. (2004. Anal. Biochem. 325: 301-307).

TABLE 5
Affinity constant of Robo-Fc L1 with human
Slit2-D2 by SPR (steady-state analysis)

| Protein | K_{D} (nM) |
|---|---|
| Robo1-Fc L1 | 32 |

A second method, which consists in determining the affinity constant between the Robo1-Fc L1 fusion protein and the human Slit2 protein, was analysed after having bound the Slit2-D2 protein to the CM5 chip. The kinetic measurements are carried out according to the protocol of Canziani et al. (2004. Anal. Biochem. 325: 301-307) using the Scatchard method according to a model with two non-equivalent binding sites.

TABLE 6
Affinity constant of Robo1-Fc with human Slit2-D2
by SPR according to the two-phase Scatchard model

| Robo1-Fc L1 | K_{D} (nM) |
|---|---|
| High-affinity site | K_{D1}: 1.5 |
| Low-affinity site | K_{D2}: 160 |

f. Affinity of Robo1-Fc for Slit measured by FACS

This example describes the affinity of the Robo1-Fc protein on HEK293 mammalian cells expressing Slit2.

The HEK293 cells described and used as in example 1 were transfected either with a ballast plasmid, having no cDNA that encodes in the mammalian cell, or the pXL4911 plasmid encoding the Slit2-D2 protein, or the pXL4912 plasmid encoding the Slit2-D1D2 protein, or pXL5033 encoding the Slit2-N protein described in example 2. The cells were distributed into 96-well plates 48 hours post-transfection, and the Robo1-Fc protein was added in a concentration range of from 0.01 to 3□mg/L for 30 min at 4° C. The Robo1-Fc protein is either the Robo1-Fc L1 biotherapeutic agent, or the Robo1-Fc Slit2-minus mutant, or the Robo1-Fc heparin-minus mutant. The cells were washed and the anti-human Fc antibody labelled with Alexa 488 (ref: A-11013, Invitrogen) was incubated for 30 min at 4° C. The labelled HEK293 cells are then quantified by FACS (Geomean).

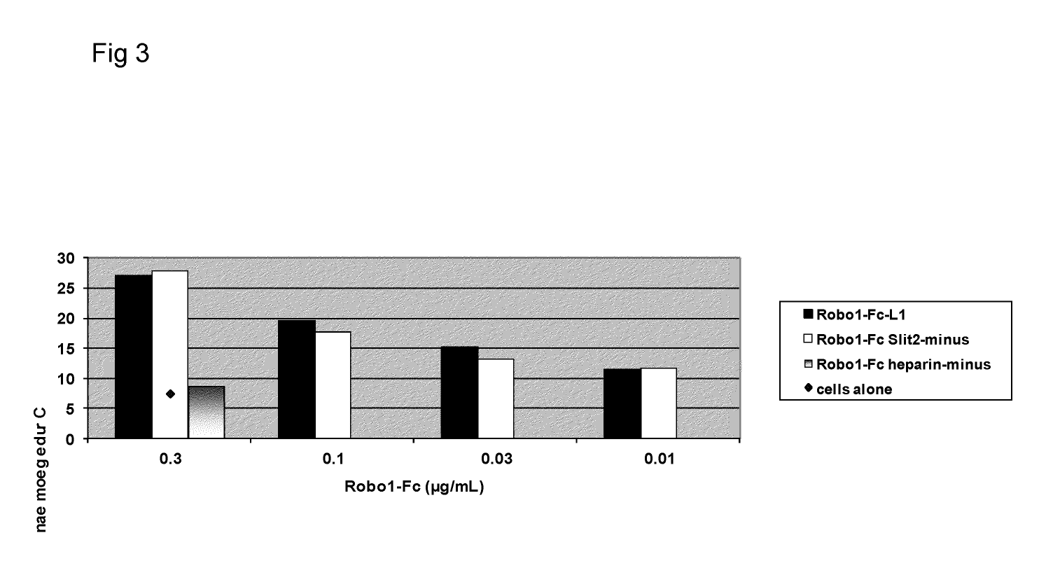

FIG. 3 describes the binding of the HEK293 cells to the Robo1-Fc protein, via the fluorescence measured by the FACS in the absence of Slit2 expression. The Robo1-Fc protein and also the Robo1-Fc Slit2-minus mutant bind to the HEK293 cells, whereas the Robo1-Fc heparin-minus mutant does not bind. Robo1-Fc therefore binds partly to the HEK293 cells via heparin binding at the low Robo1-Fc concentrations of 0.3 to 0.03 mg/L.

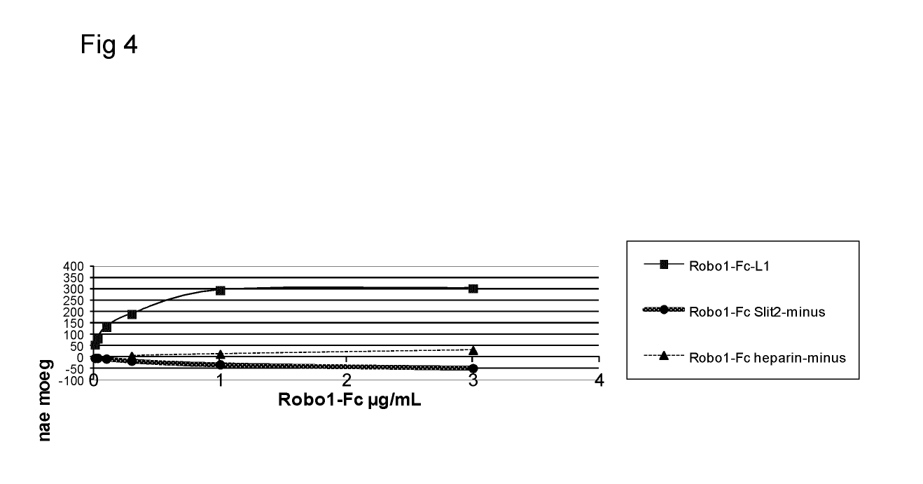

FIG. 4 describes the binding of the HEK293 cells to the Robo1-Fc protein, via the fluorescence measured by FACS when Slit2-N is expressed by transient transfection. Only the Robo1-Fc protein binds to the HEK293 cells expressing Slit2-N. The Robo1-Fc Slit2-minus and Robo1-Fc heparin-minus mutants do not bind (or virtually not) in the Robo1-Fc concentration range of 3.0 to 0.3 mg/L, compared with the biotherapeutic Robo1-Fc L1 protein.

Table 7 describes the affinity constants measured by FACS for the Robo1-Fc protein when the Slit2-N, Slit2-D1 D2 or Slit2-D2 proteins are expressed in the HEK293 line.

TABLE 7
Affinity of Robo1-Fc for the Slit2 protein on cells by FACS

| | Protein expressed | |
|---|---|---|
| Ligand Robo1-Fc | transiently in HEK293 | K_{D} (nM) |
| Robo1-Fc L1 | Slit2-N | 1.2 |
| | Slit2-D1D2 | 0.98 |
| | Slit2-D2 | Very weak binding |
| Robo1-Fc Heparin-minus | Slit2-N | 43 |
| | Slit2-D1D2 | 41 |
| Robo1-Fc Slit2-minus | Slit2-N | No binding |

As in the previous examples, the Robo1-Fc Slit2-minus mutant proved to be Slit2-negative and the Robo1-Fc heparin-minus mutant has a weaker affinity for Slit2 than the biotherapeutic protein.

Robo1-Fc binds to Slit2-N and Slit2-D1D2 expressed by the HEK293 cells with comparable affinities which are better than the affinity with Slit2-D2.

### Example 4

### Pharmacokinetic Properties of the Robo1-Fc L1 and Robo1-Fc L2 Proteins

This example describes the pharmacokinetic profile and the plasma concentration of the Robo1-Fc protein injected once in mice intravenously (iv).

Three Balb/C mice (for each time) were injected, via the caudal vein, with each of the Robo1-Fc proteins at 2.5 mg/mL in a proportion of 100 μL/10 g (≈25 mg/kg). At the predetermined times (0.5, 1, 6, 24, 48 and 72 h after administration), the mice were anaesthetized, and blood was sampled and collected in a tube containing 10 μL of citrate (CPD-A, C-4431 Sigma) and 2 μL of protease inhibitors (Complete Protease Inhibitor Mix, Roche Molecular Biochemical). The tubes were centrifuged and the plasma samples were collected and frozen at −20° C.

The wells of 96-well plates were coated with the Slit2 protein (Slit2-D2), and the plasma samples, diluted to 1/5000 and 1/20 000, were brought into contact for one hour at 37° C. The peroxidase-conjugated anti-human Fc antibody (ref. 31413, Pierce) was subsequently incubated and then visualized with TMB-H₂O₂ (ref UP664780, Interchim) and the absorbance was read at 450 nm. A calibration range was prepared with each purified Robo1-Fc protein.

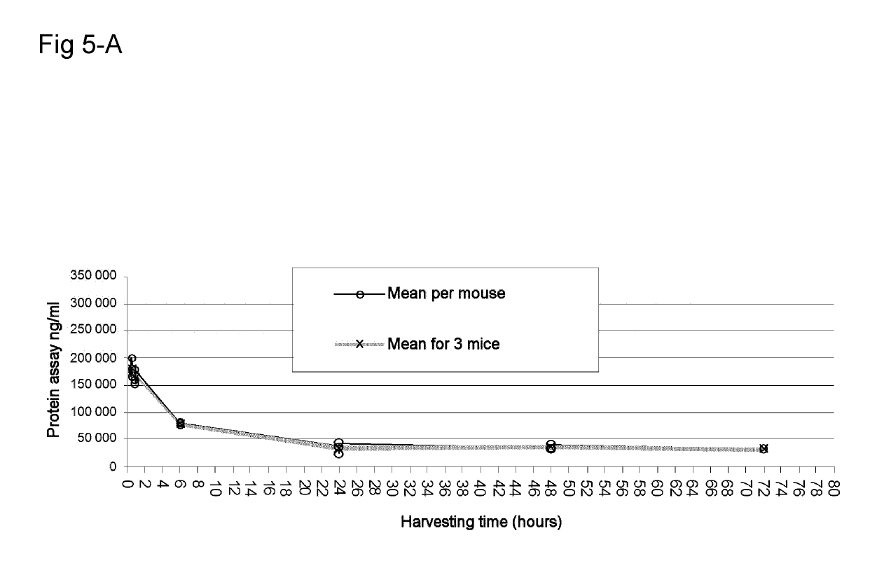

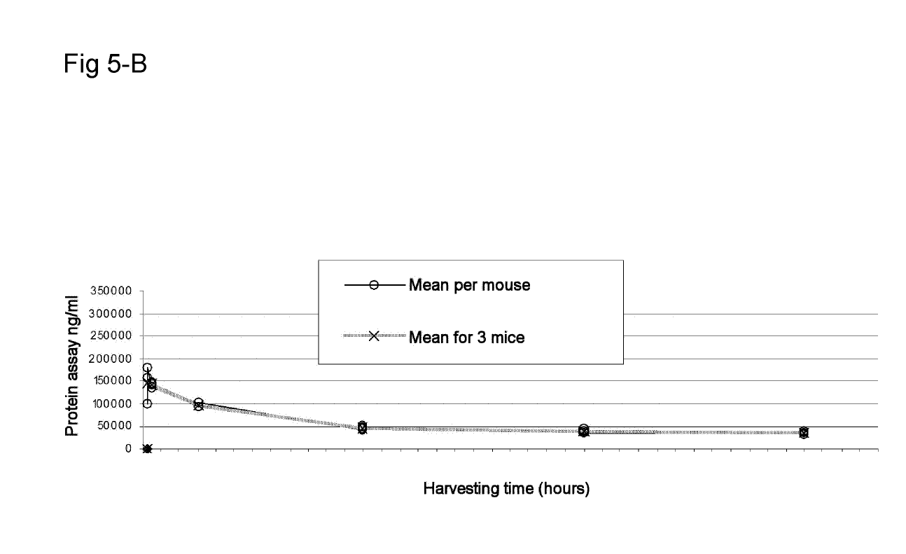

The plasma concentrations of the Robo1-Fc L1 and Robo1-Fc L2 proteins are represented in FIG. 5. The pharmacokinetic parameters are described in the following table 8 and show that the protein is stable after injection in mice.

TABLE 8
Pharmacokinetic parameters of the Robo1-Fc
proteins after iv injection in mice

| | | | AUC | AUC | | |
|---|---|---|---|---|---|---|
| Protein at | C₀ | T_{1/2} | (0-last) | (0-24 h) | CI | Vdss |
| 25 mg/kg | μg/mL | h | μg · h/mL | μg · h/mL | mL/H/kg | mL/kg |
| Robo1-Fc | 203 | 158 | 3655 | 1879 | 2.2 | 470 |
| L1 | | | | | | |
| Robo1-Fc | 198 | 142 | 3973 | 2042 | 2.2 | 417 |
| L2 | | | | | | |

### Example 5

### Description of the Robo1-Fc Biotherapeutic Protein Improved with Respect to its Homogeneity in the N-Terminal Position

This example describes the expression plasmid, the production and the physicochemical characterization of another Robo1-Fc protein, called Robo1-Fc L3, which is different from the Robo1-Fc L1 protein by virtue of the absence of the first two residues Ser20 and Arg21.

The cDNA cloned into the pXL4904 plasmid was modified by PCR in order to eliminate the Ser20 and Arg21 codons, and to fuse the next codon (Leu22) to the coding sequence of the peptide signal of interleukin 2. The pXL5004 expression plasmid was then generated, see FIG. 1. The cDNA sequence used to express this recombinant protein corresponds to the sequence SEQ ID NO.23.

The Robo1-Fc L3 protein was produced, purified and characterized as described in example 1. The N-terminal analysis showed that this purified protein was perfectly homogeneous. The recombinant protein obtained is called Robo1-Fc L3 and corresponds to the protein sequence SEQ ID NO.24.

### Example 6

### Evaluation of the Interaction of the Robo1-Fc L3 Protein with the Human Slit2 Protein

This example describes the interaction of the Robo1-Fc L3 fusion protein with the human Slit2 protein (Slit2-D2) by ELISA assay.

The human Slit2-D2 protein was bound to Immulon-4 enzyme-linked plates (VWR Scientific Inc. Swedesboro, N.J.). A concentration range (from 1 μg/mL to 0.001 μg/mL) of the Robo1-Fc L3 fusion protein was added and then detected by means of the peroxidase-conjugated goat anti-human IgG antibody (Sigma; ref. A0170, dilution to 1:50 000). The visualization was carried out with the TMB-H₂O₂ substrate (Interchim; ref UP664780) and the measurements were carried out with a plate reader at 450 nm. The results obtained are reported in table 9 below.

TABLE 9
Affinity of the Robo1-Fc L3 protein for the Slit2
protein - Comparison with the Robo1-Fc L1 protein

| Proteins studied | EC50 (μg/ml) | CV (%) |
|---|---|---|
| Robo1-Fc L1 | 0.13 | 3.3 |
| Robo1-Fc L3 | 0.17 | 4.4 |

These results show that the affinities of the two variants Robo1-Fc L1 and Robo1-Fc L3 for the Slit2-D2 protein are comparable.

### Example 7

### Evaluation of the Activity of the Robo-Fc Protein on Neovascularization

a. In Vitro Endothelial Cell and Fibroblast Coculture Model: Specific Activity of the Robo1-Fc L1 Molecule

The in vitro angiogenesis model corresponds to a rearrangement of human vein endothelial cells on a monolayer of human dermal fibroblasts. Briefly, the fibroblasts (Lonza) are seeded into 24-well plates (Becton Dickinson) at 40 000 cells/well in 1 ml of medium. After 3 days of proliferation (D3), human vein endothelial cells (HUVEC-Lonza) are seeded onto the fibroblast cell monolayer at 10 000 cells/well in 500 μl of EGM® medium (endothelial basal medium, Lonza)+2% FCS (foetal calf serum−Lonza)+10 μg/ml hEGF (recombinant human epidermal growth factor−Lonza). The cells are stimulated with 30 ng/mL of VEGF (R&D Systems), with the Robo1-Fc L1 molecule or with a Robo1-Fc Slit2-minus negative control at the concentration of 500 μg/ml (D3 to D9). After 3 days, the medium is replaced and the wells are stimulated according to the conditions of the experiment.

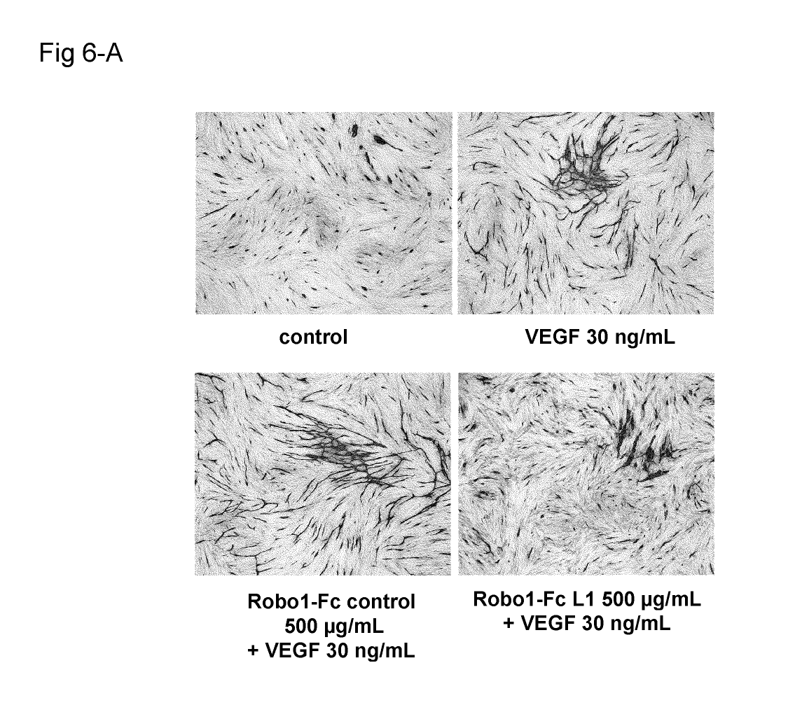

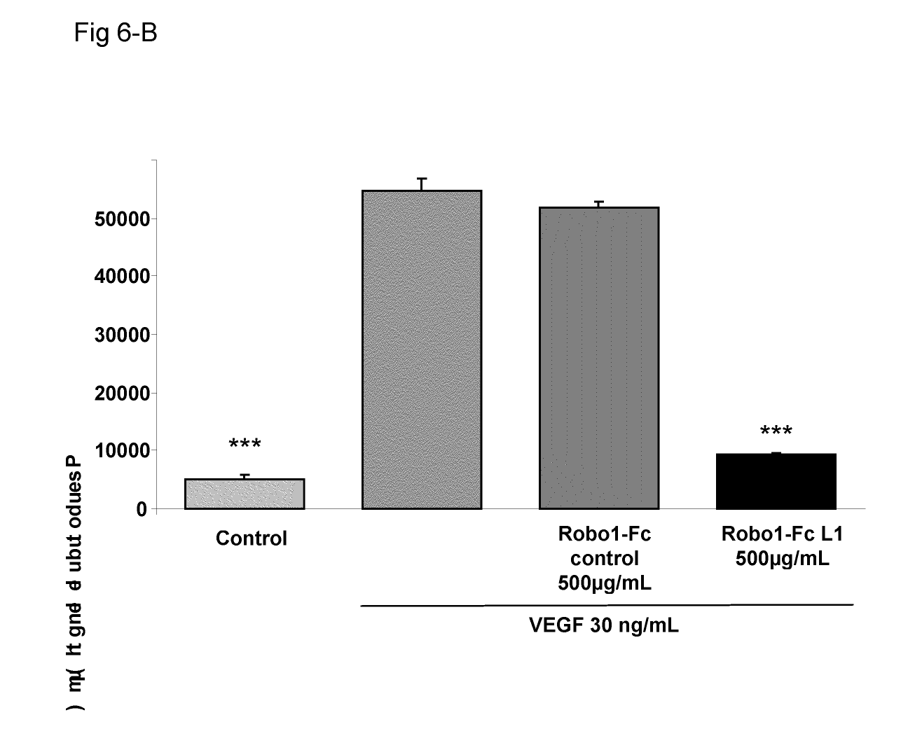

After 2 days, the cells are fixed with ethanol and labelled with an anti-CD31 antibody specific for HUVECs, followed by an anti-alkaline phosphatase antibody, and then visualized with an alkaline phosphatase substrate (D11). A quantification of the tubules labelled with the anti-CD31 antibody is carried out by means of image acquisitions made under a microscope (×4 objective) and the length of the pseudotubules is analysed using image analysis software (Biocom Visiolab 2000 software) (FIG. 6).

In this in vitro angiogenesis assay, Robo1-Fc L1 (500 μg/ml) shows an inhibitory activity on the formation of the tubules formed by the HUVECs. This inhibition amounts to 82% and it is statistically significant compared with the effect obtained with the Robo1-Fc Slit2-minus molecule (negative control).

b. Ex Vivo Mouse Aortic Ring Model: Specific Activity of the Robo1-Fc L1 Molecule

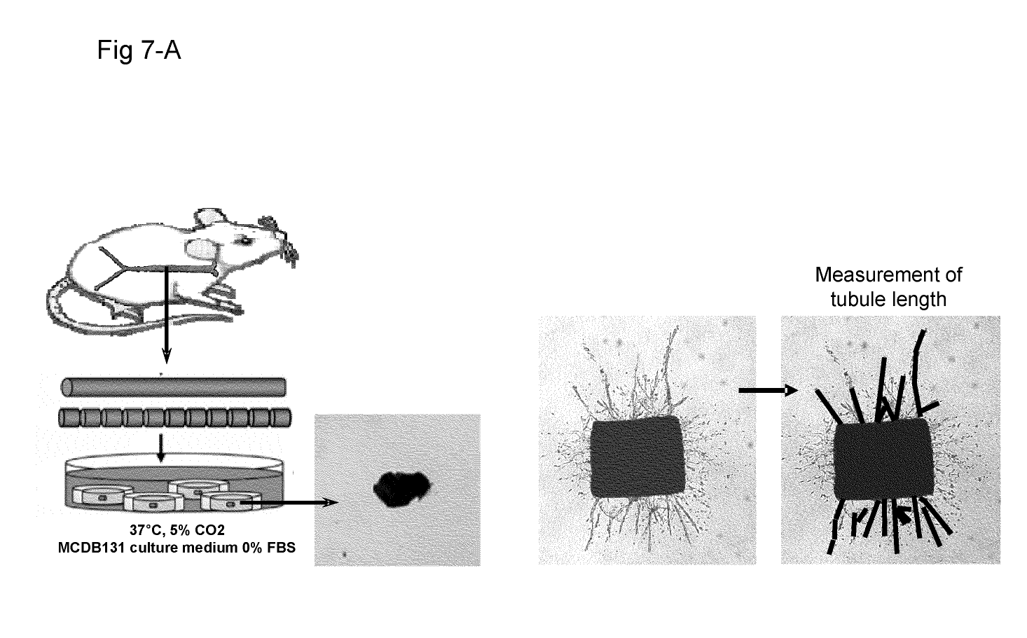

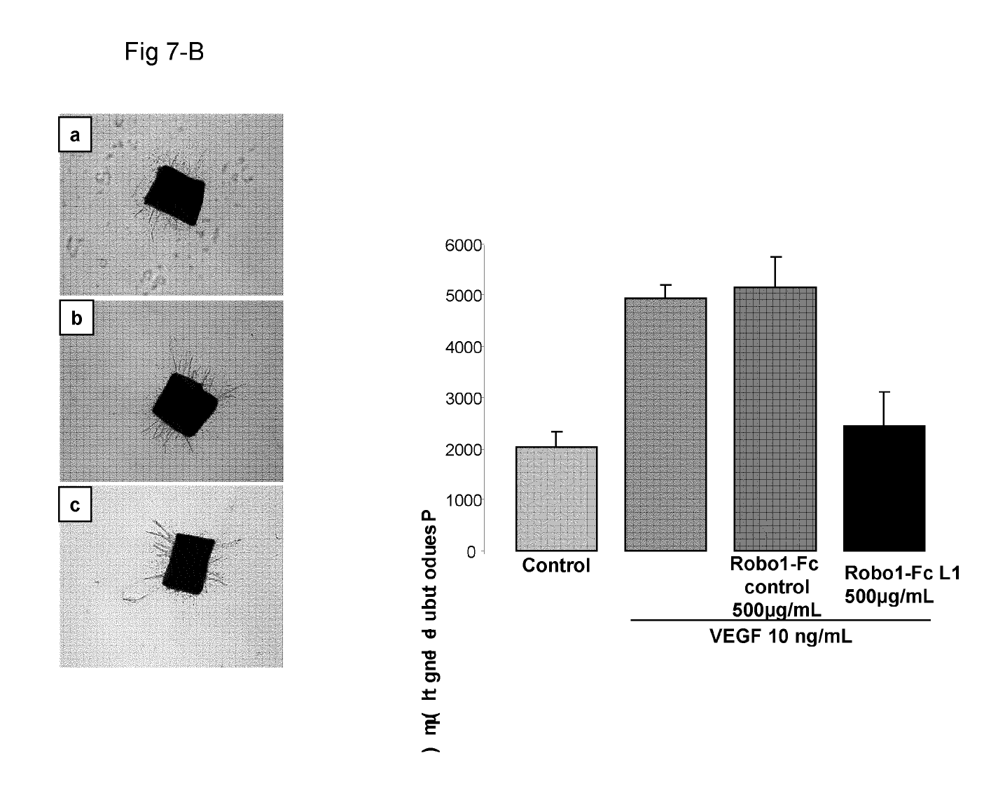

The Robo1-Fc L1 molecule was evaluated in a mouse aortic ring model. Briefly, mouse aortas are removed and cleaned, and cut into sections of 1 mm (D0). These rings are embedded in rat collagen in the presence of VEGF at 10 ng/ml, of the Robo1-Fc L1 molecule at the concentration of 500 μg/ml or of a Robo1-Fc Slit2-minus negative control at the concentration of 500 μg/ml. Tubules will form from the ring, thus mimicking in vitro, the formation of neovessels. After 6 days, a quantification of the labelled tubules is carried out by means of image acquisitions made under a microscope (×3 objective) (FIG. 7A) and the length of the pseudotubules is analysed using image analysis software (Biocom Visiolab software 2000) (FIG. 7).

Under these experimental conditions, Robo1-Fc L1 (500 μg/ml) shows a strong inhibitory activity on the formation of the tubules formed, in comparison with the Robo1-Fc Slit2-minus molecule used as a negative control.

These results suggest that Robo1-Fc L1 is capable of inhibiting the formation of neovessels without inhibiting endothelial cell proliferation. This anti-angiogenic activity linked to a vessel maturation defect is called “non-productive angiogenesis”.

### Example 8

### Evaluation of the Robo1-Fc L1 Protein in a Lung Tumour Model in Mice

The Robo1-Fc L1 molecule was evaluated in a model of a lung cancer tumour in C57/BI6 mice.

a. Murine Lung Tumour Model

In order to establish the murine lung tumour model, 8-week-old female C57/Bl6 mice were anaesthetized. The area at the level of the left scapula of the mouse was shaved and disinfected. A 1 cm incision was made above the scapula.

The cells to be injected are derived from a Lewis lung carcinoma tumour line (ATCC, CRL-1642). The cells were mixed with Matrigel® in a ratio of 1 vol of Matrigel to 4 vol of cells. The cell concentration was 62 500 cells/ml. The cells were injected into the lung at a rate of 20 μl per mouse, and then the wound was sutured.

After 23 days, the mice were euthanized. The ribcage was opened up, and the left lung and the mediastinal lymph nodes were removed. The tumour present on the left lung was measured using an electronic calliper rule in order to determine the tumour volume according to the formula: I2×L×0.52. The mediastinal lymph nodes are weighed. The results are expressed as mean value±standard deviation from the mean. The statistical analysis was carried out by means of a parametric Student's test.

b. Treatment of the Mice Bearing a Lung Tumour with the Robo1-Fc Recombinant Protein

The treatment using the Robo1-Fc protein was carried out as follows: a preparation containing the Robo1-Fc protein was injected at the dose of 25 mg/kg/day, intravenously, on D10, D14, D17 and D21 post-injection of the tumour cells. The control group was injected with PBS buffer (10 ml/kg).

c. Results

On D23, the mean volume of the tumours obtained in the group treated with the Robo1-Fc recombinant protein was 21.45±2.16 mm³; the mean volume of the tumours obtained in the control group was 39.93±8.41 mm³. The reduction in tumour volume in the animals treated with the Robo1-Fc protein is 46%. This difference is statistically significant (p<0.05). The mean weight of the mediastinal lymph nodes (metastatic lymph nodes) obtained in the group treated with the Robo1-Fc protein is 12.50±1.26 mg. The mean weight of the mediastinal lymph nodes obtained in the control group is 30.67±7.69 mg. The reduction in weight of the mediastinal lymph nodes for the group treated with the Robo1-Fc protein is 59% at the limit of significance (p=0.07).

### Example 9

### Evaluation of the Robo1-Fc 1:1 Protein in Two Models of Human Hepatocarcinoma

The Robo1-Fc L1 molecule was evaluated in two models of human hepatocarcinoma in SCID mice.

A. Orthotopic Model of Human Hepatocarcinoma in SCID Mice Using HepG2 Cell Line

a. Description of the Model

Female SCID mice are anaesthetized (with a mixture of ketamine/xylazine) and the area of the incision is shaved. The skin is cleaned and disinfected before making a 1 cm subcostal incision in the skin and in the muscle wall. A part of the lobe of the liver is carefully extracted from the peritoneum in order to carry out the injection of the cells. A 50 μl volume of the cell suspension (HepG2 originating from ATCC, human hepatoblastoma cells mixed with Matrigel® at 40×10⁶ cells/ml) is injected into the left lobe. The lobe is put back into the abdominal cavity. The peritoneum is closed with surgical glue and then the skin is sutured with staples. The mice are treated with Robo1-Fc solubilized in PBS at 25 mg/kg, i.p. injection twice a week. The treatment begins two weeks after the implantation of the cells. Four weeks after inoculation with the cells, the animals are sacrificed for autopsy.

b. Treatment of the Mice Bearing a Human Hepatocarcinoma with the Robo1-Fc Recombinant Protein

Robo1-Fc was evaluated in this model with a treatment twice a week at the dose of 25 mg/kg. The weight of the mice is measured on the first and last day of the protocol. The weight of the tumours was measured 28 days after the injection of the cells, after sacrificing the animals.

c. Results

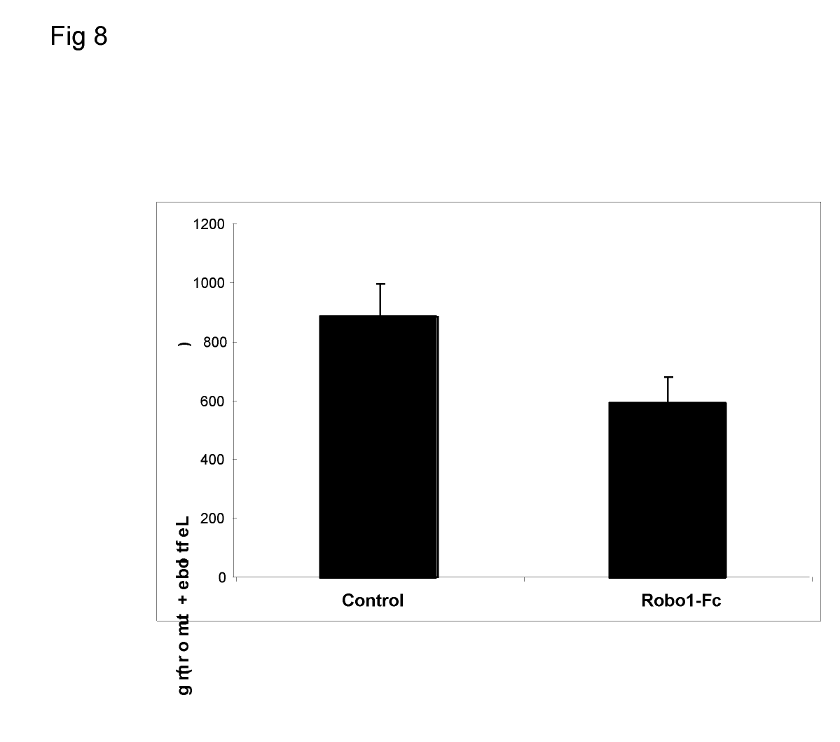

The tumour growth leads to a considerable loss of weight by the control animals during the protocol, whereas the animals treated with Robo1-Fc have an identical weight at the end of the experiment (Table 10). At the end of the experiment, after 4 i.p. injections of Robo1-Fc, the mean tumour weight of the Robo-1-Fc-treated group is significantly reduced by 30% (p<0.05) in comparison with the control group having received injections of solvent (FIG. 8).

TABLE 10
Weight of animals at the beginning and end of the protocol

Mean weight of mice (g)
Mean (+/−standard deviation of the mean)

| | D 0 | D 28 |
|---|---|---|
| Control (n = 11) | 19.24 ± 0.48 | 16.46 ± 0.54 |
| Robo1-Fc (n = 13) | 19.17 ± 0.31 | 19.07 ± 0.63 |

B. Orthotopic Model of Human Hepatocarcinoma in SCID Mice Using Hep3B Cell Line

a. Description of the Model

In this HCC model, Hep3B cell line was used. This tumor cell line is a commercial cell line (from ATCC) coming from an hepatoblastoma of a young patient. The same protocol of cell injection was used as previously described in example 9A a). The initial concentration of cells is 2.10⁶ in an injected volume of 50 μl.

b. Treatment of Mice Bearing Hep3B Tumors with Robo1-Fc

Robo1-Fc was evaluated in this model by a treatment twice a week at 5 mg/kg. The mice were weighted before treatment and each time before the next treatment in order to adjust the dosage with the injected volume. The mice were also weighted the last day of the protocol, before sacrifice. The lobe with the tumor was measured after sacrifice on the last day of the protocol. Furthermore, before sacrifice, the blood of mice were withdrawn and the serum was discarded for afetoprotein (AFP) determination; AFP is secreted by the cancer liver cells and it is currently accepted as a biomarker of HCC development in humans (Fimus et al., 2004, Mol Diagn8(4):207-12. Review).

c. Results

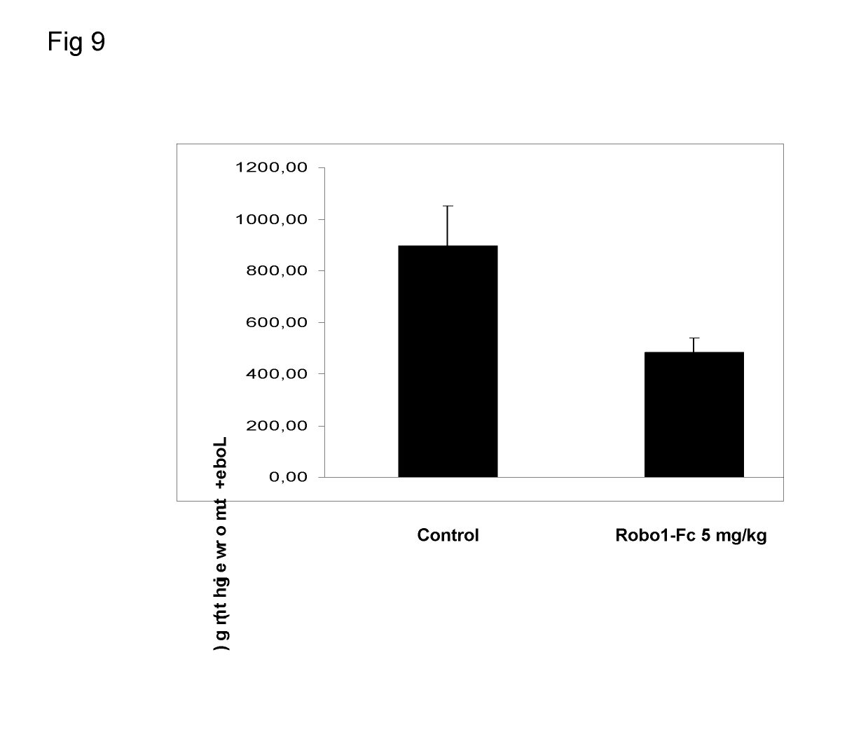

The mice did not gain weight during the tumoral development for 35 days. The treatment of Robo1-Fc at 5 mg/kg showed no effect on the mice weight (Table 11). At the end of the experiment, the mean tumoral weight of the control group was 895±156 mg. the mean tumoral weight of the Robo1-Fc treated group was 483±55 mg (FIG. 9). Treatment with Robo1-Fc induced a significant reduction of the tumor burden of 46%.

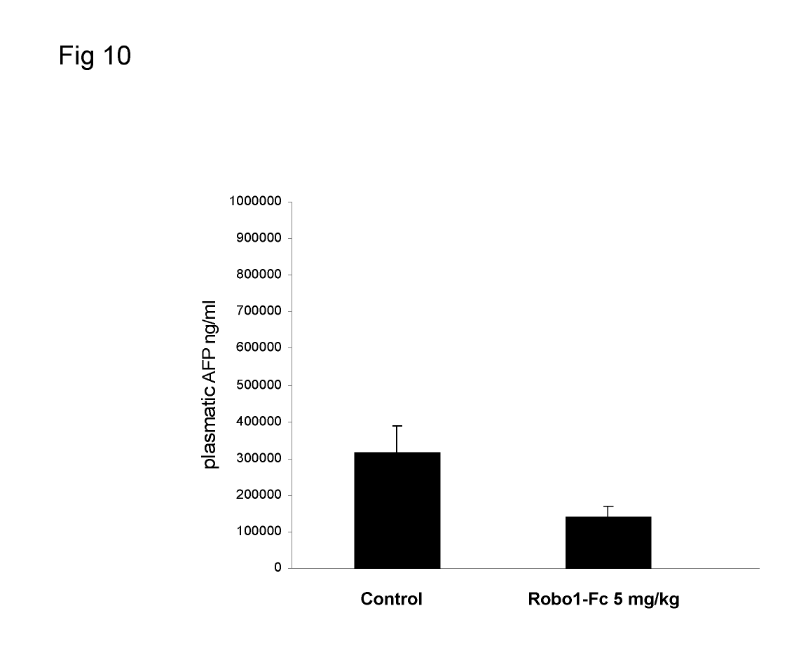

Furthermore the plasmatic AFP concentration was 316±73 μg/ml in control tumor bearing mice and 139±28 μg/ml in Robo1-Fc treated mice, showing a 56% inhibition of plasmatic AFP in treated animals (FIG. 10). This inhibition of plasmatic AFP correlated well with the anti tumoral activity of Robo1-Fc.

TABLE 11
Mice weight at the beginning and at the end of the protocol

Mice weight
Mean +/− sem

| | D 0 | D 35 |
|---|---|---|
| Control group (n = 14) | 19.08 ± 0.30 | 19.12 ± 0.76 |
| Robo1-Fc group (n = 14) | 19.36 ± 0.36 | 20.37 ± 0.31 |

1. A method for treating cancer in a subject suffering therefrom, comprising administering to the subject a recombinant protein comprising the extracellular domain derived from Robo1 protein or a part of this domain, thereby treating said cancer.

2. The method according to claim 1, wherein said cancer is hepatocarcinoma.

3. The method according to claim 1, wherein said recombinant protein does not inhibit endothelial cell proliferation.

4. The method according to claim 1, wherein said extracellular domain is an extracellular domain derived from isoform b of the Robo1 protein.

5. The method according to claim 4, in which the extracellular domain of Robo1 or part thereof consists in the Ig1 and Ig2 immunoglobulin domains of said extracellular domain.

6. The method according to claim 5, in which the extracellular domain of Robo1 has at least 80% identity with the sequence SEQ ID NO.2.

7. The method according to claim 1, wherein said protein further comprises a linker and an immunoglobulin Fc domain.

8. The method according to claim 7, in which the Fc domain comes from human immunoglobulin G4.

9. The method according to claim 8, in which the Fc domain contains the S241P and L248E mutations and in which the lysine located in the C-terminal position is absent.

10. The method according to claim 1, wherein the sequence of said protein has at least 80% identity with the sequence SEQ ID NO. 4, SEQ ID NO.6 or SEQ ID NO. 24.

11. The method according to claim 1, wherein the sequence of said protein comprises the sequence SEQ ID NO. 4, SEQ ID NO.6 or SEQ ID NO. 24.


SEQUENCE LISTING (24 sequences)

SEQ ID NO: 1 - DNA, 660 bp, Homo sapiens
  CDS (1)..(660)
atgattgcggagcccgctcacttttacctgtttggattaatatgtctctgttcaggctcccgtcttcgtcaggaagattttccacctcgcattgttgaacacccttcagacctgattgtctcaaaaggagaacctgcaactttgaactgcaaagctgaaggccgccccacacccactattgaatggtacaaagggggagagagagtggagacagacaaagatgaccctcgctcacaccgaatgttgctgccgagtggatctttatttttcttacgtatagtacatggacggaaaagtagacctgatgaaggagtctatgtctgtgtagcaaggaattaccttggagaggctgtgagccacaatgcatcgctggaagtagccatacttcgggatgacttcagacaaaacccttcggatgtcatggttgcagtaggagagcctgcagtaatggaatgccaacctccacgaggccatcctgagcccaccatttcatggaagaaagatggctctccactggatgataaagatgaaagaataactatacgaggaggaaagctcatgatcacttacacccgtaaaagtgacgctggcaaatatgtttgtgttggtaccaatatggttggggaacgtgagagtgaagtagccgagctgactgtctta

SEQ ID NO: 2 - protein, 220 aa, Homo sapiens
MIAEPAHFYLFGLICLCSGSRLRQEDFPPRIVEHPSDLIVSKGEPATLNCKAEGRPTPTIEWYKGGERVETDKDDPRSHRMLLPSGSLFFLRIVHGRKSRPDEGVYVCVARNYLGEAVSHNASLEVAILRDDFRQNPSDVMVAVGEPAVMECQPPRGHPEPTISWKKDGSPLDDKDERITIRGGKLMITYTRKSDAGKYVCVGTNMVGERESEVAELTVL

SEQ ID NO: 3 - DNA, 1365 bp, Homo sapiens
  CDS (1)..(1365)
atgattgcggagcccgctcacttttacctgtttggattaatatgtctctgttcaggctcccgtcttcgtcaggaagattttccacctcgcattgttgaacacccttcagacctgattgtctcaaaaggagaacctgcaactttgaactgcaaagctgaaggccgccccacacccactattgaatggtacaaagggggagagagagtggagacagacaaagatgaccctcgctcacaccgaatgttgctgccgagtggatctttatttttcttacgtatagtacatggacggaaaagtagacctgatgaaggagtctatgtctgtgtagcaaggaattaccttggagaggctgtgagccacaatgcatcgctggaagtagccatacttcgggatgacttcagacaaaacccttcggatgtcatggttgcagtaggagagcctgcagtaatggaatgccaacctccacgaggccatcctgagcccaccatttcatggaagaaagatggctctccactggatgataaagatgaaagaataactatacgaggaggaaagctcatgatcacttacacccgtaaaagtgacgctggcaaatatgtttgtgttggtaccaatatggttggggaacgtgagagtgaagtagccgagctgactgtcttagagagaccatcatttgtggagtccaagtacggccctccttgccctccctgccctgcccctgagttcgagggcggacctagcgtgttcctgttccctcctaagcctaaggacaccctgatgatctcccggacccctgaggtgacctgtgtggtggtggacgtgtcccaggaggaccctgaggtccagttcaactggtacgtggacggcgtggaggtgcacaacgccaagaccaagcctcgggaggagcagttcaattccacctaccgggtggtgtctgtgctgaccgtgctgcaccaggactggctgaacggcaaagaatacaagtgtaaggtctccaacaagggcctgccctcctccatcgagaaaaccatctccaaggccaagggccagcctagggagcctcaggtgtacaccctgcctcctagccaggaagagatgaccaagaaccaggtgtccctgacctgtctggtgaagggcttctacccttccgacatcgccgtggagtgggagtccaacggccagcctgagaacaactacaagaccacccctcctgtgctggactccgacggctccttcttcctgtactccaggctgaccgtggacaagtcccggtggcaggagggcaacgtcttttcctgctccgtgatgcacgaggccctgcacaaccactacacccagaagtccctgtccctgtctctgggctga

SEQ ID NO: 4 - protein, 454 aa, Homo sapiens
MIAEPAHFYLFGLICLCSGSRLRQEDFPPRIVEHPSDLIVSKGEPATLNCKAEGRPTPTIEWYKGGERVETDKDDPRSHRMLLPSGSLFFLRIVHGRKSRPDEGVYVCVARNYLGEAVSHNASLEVAILRDDFRQNPSDVMVAVGEPAVMECQPPRGHPEPTISWKKDGSPLDDKDERITIRGGKLMITYTRKSDAGKYVCVGTNMVGERESEVAELTVLERPSFVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 5 - DNA, 1362 bp, Homo sapiens
  CDS (1)..(1362)
atgattgcggagcccgctcacttttacctgtttggattaatatgtctctgttcaggctcccgtcttcgtcaggaagattttccacctcgcattgttgaacacccttcagacctgattgtctcaaaaggagaacctgcaactttgaactgcaaagctgaaggccgccccacacccactattgaatggtacaaagggggagagagagtggagacagacaaagatgaccctcgctcacaccgaatgttgctgccgagtggatctttatttttcttacgtatagtacatggacggaaaagtagacctgatgaaggagtctatgtctgtgtagcaaggaattaccttggagaggctgtgagccacaatgcatcgctggaagtagccatacttcgggatgacttcagacaaaacccttcggatgtcatggttgcagtaggagagcctgcagtaatggaatgccaacctccacgaggccatcctgagcccaccatttcatggaagaaagatggctctccactggatgataaagatgaaagaataactatacgaggaggaaagctcatgatcacttacacccgtaaaagtgacgctggcaaatatgtttgtgttggtaccaatatggttggggaacgtgagagtgaagtagccgagctgactgtcttaggaggcggaggatccgagtccaagtacggccctccttgccctccctgccctgcccctgagttcgagggcggacctagcgtgttcctgttccctcctaagcctaaggacaccctgatgatctcccggacccctgaggtgacctgtgtggtggtggacgtgtcccaggaggaccctgaggtccagttcaactggtacgtggacggcgtggaggtgcacaacgccaagaccaagcctcgggaggagcagttcaattccacctaccgggtggtgtctgtgctgaccgtgctgcaccaggactggctgaacggcaaagaatacaagtgtaaggtctccaacaagggcctgccctcctccatcgagaaaaccatctccaaggccaagggccagcctagggagcctcaggtgtacaccctgcctcctagccaggaagagatgaccaagaaccaggtgtccctgacctgtctggtgaagggcttctacccttccgacatcgccgtggagtgggagtccaacggccagcctgagaacaactacaagaccacccctcctgtgctggactccgacggctccttcttcctgtactccaggctgaccgtggacaagtcccggtggcaggagggcaacgtcttttcctgctccgtgatgcacgaggccctgcacaaccactacacccagaagtccctgtccctgtctctgggctga

SEQ ID NO: 6 - protein, 453 aa, Homo sapiens
MIAEPAHFYLFGLICLCSGSRLRQEDFPPRIVEHPSDLIVSKGEPATLNCKAEGRPTPTIEWYKGGERVETDKDDPRSHRMLLPSGSLFFLRIVHGRKSRPDEGVYVCVARNYLGEAVSHNASLEVAILRDDFRQNPSDVMVAVGEPAVMECQPPRGHPEPTISWKKDGSPLDDKDERITIRGGKLMITYTRKSDAGKYVCVGTNMVGERESEVAELTVLGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 7 - DNA, 1365 bp, Homo sapiens
  CDS (1)..(1365)
atgattgcggagcccgctcacttttacctgtttggattaatatgtctctgttcaggctcccgtcttcgtcaggaagattttccacctcgcattgttgaacacccttcagaccagattgtctcaaaaggagaacctgcaactttgaactgcaaagctgaaggccgccccacacccactattgaatggtacaaagggggagagagagtggagacagacaaagatgaccctcgctcacaccgaatgttgctgccgagtggatctttatatttcttacgtatagtacatggacggaaaagtagacctgatgaaggagtctatgtctgtgtagcaaggaattaccttggagaggctgtgagccacaatgcatcgctggaagtagccatacttcgggatgacttcagacaaaacccttcggatgtcatggttgcagtaggagagcctgcagtaatggaatgccaacctccacgaggccatcctgagcccaccatttcatggaagaaagatggctctccactggatgataaagatgaaagaataactatacgaggaggaaagctcatgatcacttacacccgtaaaagtgacgctggcaaatatgtttgtgttggtaccaatatggttggggaacgtgagagtgaagtagccgagctgactgtcttagagagaccatcatttgtggagtccaagtacggccctccttgccctccctgccctgcccctgagttcgagggcggacctagcgtgttcctgttccctcctaagcctaaggacaccctgatgatctcccggacccctgaggtgacctgtgtggtggtggacgtgtcccaggaggaccctgaggtccagttcaactggtacgtggacggcgtggaggtgcacaacgccaagaccaagcctcgggaggagcagttcaattccacctaccgggtggtgtctgtgctgaccgtgctgcaccaggactggctgaacggcaaagaatacaagtgtaaggtctccaacaagggcctgccctcctccatcgagaaaaccatctccaaggccaagggccagcctagggagcctcaggtgtacaccctgcctcctagccaggaagagatgaccaagaaccaggtgtccctgacctgtctggtgaagggcttctacccttccgacatcgccgtggagtgggagtccaacggccagcctgagaacaactacaagaccacccctcctgtgctggactccgacggctccttcttcctgtactccaggctgaccgtggacaagtcccggtggcaggagggcaacgtcttttcctgctccgtgatgcacgaggccctgcacaaccactacacccagaagtccctgtccctgtctctgggctga

SEQ ID NO: 8 - protein, 454 aa, Homo sapiens
MIAEPAHFYLFGLICLCSGSRLRQEDFPPRIVEHPSDQIVSKGEPATLNCKAEGRPTPTIEWYKGGERVETDKDDPRSHRMLLPSGSLYFLRIVHGRKSRPDEGVYVCVARNYLGEAVSHNASLEVAILRDDFRQNPSDVMVAVGEPAVMECQPPRGHPEPTISWKKDGSPLDDKDERITIRGGKLMITYTRKSDAGKYVCVGTNMVGERESEVAELTVLERPSFVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 9 - DNA, 1365 bp, Homo sapiens
  CDS (1)..(1365)
atgattgcggagcccgctcacttttacctgtttggattaatatgtctctgttcaggctcccgtcttcgtcaggaagattttccacctcgcattgttgaacacccttcagacctgattgtctcaaaaggagaacctgcaactttgaactgcaaagctgaaggccgccccacacccactattgaatggtacaaagggggagagagagtggagacagacaaagatgaccctcgctcacaccgaatgttgctgccgagtggatctttatttttcttacgtatagtacatggagccgccagtagacctgatgaaggagtctatgtctgtgtagcaaggaattaccttggagaggctgtgagccacaatgcatcgctggaagtagccatacttcgggatgacttcagacaaaacccttcggatgtcatggttgcagtaggagagcctgcagtaatggaatgccaacctccacgaggccatcctgagcccaccatttcatggaagaaagatggctctccactggatgataaagatgaaagaataactatacgaggaggaaagctcatgatcacttacacccgtaaaagtgacgctggcaaatatgtttgtgttggtaccaatatggttggggaacgtgagagtgaagtagccgagctgactgtcttagagagaccatcatttgtggagtccaagtacggccctccttgccctccctgccctgcccctgagttcgagggcggacctagcgtgttcctgttccctcctaagcctaaggacaccctgatgatctcccggacccctgaggtgacctgtgtggtggtggacgtgtcccaggaggaccctgaggtccagttcaactggtacgtggacggcgtggaggtgcacaacgccaagaccaagcctcgggaggagcagttcaattccacctaccgggtggtgtctgtgctgaccgtgctgcaccaggactggctgaacggcaaagaatacaagtgtaaggtctccaacaagggcctgccctcctccatcgagaaaaccatctccaaggccaagggccagcctagggagcctcaggtgtacaccctgcctcctagccaggaagagatgaccaagaaccaggtgtccctgacctgtctggtgaagggcttctacccttccgacatcgccgtggagtgggagtccaacggccagcctgagaacaactacaagaccacccctcctgtgctggactccgacggctccttcttcctgtactccaggctgaccgtggacaagtcccggtggcaggagggcaacgtcttttcctgctccgtgatgcacgaggccctgcacaaccactacacccagaagtccctgtccctgtctctgggctga

SEQ ID NO: 10 - protein, 454 aa, Homo sapiens
MIAEPAHFYLFGLICLCSGSRLRQEDFPPRIVEHPSDLIVSKGEPATLNCKAEGRPTPTIEWYKGGERVETDKDDPRSHRMLLPSGSLFFLRIVHGAASRPDEGVYVCVARNYLGEAVSHNASLEVAILRDDFRQNPSDVMVAVGEPAVMECQPPRGHPEPTISWKKDGSPLDDKDERITIRGGKLMITYTRKSDAGKYVCVGTNMVGERESEVAELTVLERPSFVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 11 - DNA, 717 bp, Homo sapiens
  CDS (1)..(717)
atgtacaggatgcaactcctgtcttgcattgcactaagtcttgcacttgtcacgaattcattgcactgccctgccgcctgtacctgtagcaacaatatcgtagactgtcgtgggaaaggtctcactgagatccccacaaatcttccagagaccatcacagaaatacgtttggaacagaacacaatcaaagtcatccctcctggagctttctcaccatataaaaagcttagacgaattgacctgagcaataatcagatctctgaacttgcaccagatgctttccaaggactacgctctctgaattcacttgtcctctatggaaataaaatcacagaactccccaaaagtttatttgaaggactgttttccttacagctcctattattgaatgccaacaagataaactgccttcgggtagatgcttttcaggatctccacaacttgaaccttctctccctatatgacaacaagcttcagaccatcgccaaggggaccttttcacctcttcgggccattcaaactatgcatttggcccagaacccctttatttgtgactgccatctcaagtggctagcggattatctccataccaacccgattgagaccagtggtgcccgttgcaccagcccccgccgcctggcaaacaaaagaattggacagatcaaaagcaagaaattccgttgttcaggtagcgctcatcaccatcatcatcactga

SEQ ID NO: 12 - protein, 238 aa, Homo sapiens
MYRMQLLSCIALSLALVTNSLHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSGSAHHHHHH

SEQ ID NO: 13 - DNA, 1467 bp, Homo sapiens
  CDS (1)..(1467)
atgcgcggcgttggctggcagatgctgtccctgtcgctggggttagtgctggcgatcctgaacaaggtggcaccgcaggcgtgcccggcgcagtgctcttgctcgggcagcacagtggactgtcacgggctggcgctgcgcagcgtgcccaggaatatcccccgcaacaccgagagactggatttaaatggaaataacatcacaagaattacgaagacagattttgctggtcttagacatctaagagttcttcagcttatggagaataagattagcaccattgaaagaggagcattccaggatcttaaagaactagagagactgcgtttaaacagaaatcaccttcagctgtttcctgagttgctgtttcttgggactgcgaagctatacaggcttgatctcagtgaaaaccaaattcaggcaatcccaaggaaagctttccgtggggcagttgacataaaaaatttgcaactggattacaaccagatcagctgtattgaagatggggcattcagggctctccgggacctggaagtgctcactctcaacaataacaacattactagactttctgtggcaagtttcaaccatatgcctaaacttaggacttttcgactgcattcaaacaacctgtattgtgactgccacctggcctggctctccgactggcttcgccaaaggcctcgggttggtctgtacactcagtgtatgggcccctcccacctgagaggccataatgtagccgaggttcaaaaacgagaatttgtctgcagtggtcaccagtcatttatggctccttcttgtagtgttttgcactgccctgccgcctgtacctgtagcaacaatatcgtagactgtcgtgggaaaggtctcactgagatccccacaaatcttccagagaccatcacagaaatacgtttggaacagaacacaatcaaagtcatccctcctggagctttctcaccatataaaaagcttagacgaattgacctgagcaataatcagatctctgaacttgcaccagatgctttccaaggactacgctctctgaattcacttgtcctctatggaaataaaatcacagaactccccaaaagtttatttgaaggactgttttccttacagctcctattattgaatgccaacaagataaactgccttcgggtagatgcttttcaggatctccacaacttgaaccttctctccctatatgacaacaagcttcagaccatcgccaaggggaccttttcacctcttcgggccattcaaactatgcatttggcccagaacccctttatttgtgactgccatctcaagtggctagcggattatctccataccaacccgattgagaccagtggtgcccgttgcaccagcccccgccgcctggcaaacaaaagaattggacagatcaaaagcaagaaattccgttgttcaggtagcgctcatcaccatcatcatcactga

SEQ ID NO: 14 - protein, 488 aa, Homo sapiens
MRGVGWQMLSLSLGLVLAILNKVAPQACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSGHQSFMAPSCSVLHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSGSAHHHHHH

SEQ ID NO: 15 - DNA, 3390 bp, Homo sapiens
  CDS (1)..(3390)
atgcgcggcgttggctggcagatgctgtccctgtcgctggggttagtgctggcgatcctgaacaaggtggcaccgcaggcgtgcccggcgcagtgctcttgctcgggcagcacagtggactgtcacgggctggcgctgcgcagcgtgcccaggaatatcccccgcaacaccgagagactggatttaaatggaaataacatcacaagaattacgaagacagattttgctggtcttagacatctaagagttcttcagcttatggagaataagattagcaccattgaaagaggagcattccaggatcttaaagaactagagagactgcgtttaaacagaaatcaccttcagctgtttcctgagttgctgtttcttgggactgcgaagctatacaggcttgatctcagtgaaaaccaaattcaggcaatcccaaggaaagctttccgtggggcagttgacataaaaaatttgcaactggattacaaccagatcagctgtattgaagatggggcattcagggctctccgggacctggaagtgctcactctcaacaataacaacattactagactttctgtggcaagtttcaaccatatgcctaaacttaggacttttcgactgcattcaaacaacctgtattgtgactgccacctggcctggctctccgactggcttcgccaaaggcctcgggttggtctgtacactcagtgtatgggcccctcccacctgagaggccataatgtagccgaggttcaaaaacgagaatttgtctgcagtggtcaccagtcatttatggctccttcttgtagtgttttgcactgccctgccgcctgtacctgtagcaacaatatcgtagactgtcgtgggaaaggtctcactgagatccccacaaatcttccagagaccatcacagaaatacgtttggaacagaacacaatcaaagtcatccctcctggagctttctcaccatataaaaagcttagacgaattgacctgagcaataatcagatctctgaacttgcaccagatgctttccaaggactacgctctctgaattcacttgtcctctatggaaataaaatcacagaactccccaaaagtttatttgaaggactgttttccttacagctcctattattgaatgccaacaagataaactgccttcgggtagatgcttttcaggatctccacaacttgaaccttctctccctatatgacaacaagcttcagaccatcgccaaggggaccttttcacctcttcgggccattcaaactatgcatttggcccagaacccctttatttgtgactgccatctcaagtggctagcggattatctccataccaacccgattgagaccagtggtgcccgttgcaccagcccccgccgcctggcaaacaaaagaattggacagatcaaaagcaagaaattccgttgttcagctaaagaacagtatttcattccaggtacagaagattatcgatcaaaattaagtggagactgctttgcggatctggcttgccctgaaaagtgtcgctgtgaaggaaccacagtagattgctctaatcaaaagctcaacaaaatcccggagcacattccccagtacactgcagagttgcgtctcaataataatgaatttaccgtgttggaagccacaggaatctttaagaaacttcctcaattacgtaaaataaactttagcaacaataagatcacagatattgaggagggagcatttgaaggagcatctggtgtaaatgaaatacttcttacgagtaatcgtttggaaaatgtgcagcataagatgttcaagggattggaaagcctcaaaactttgatgttgagaagcaatcgaataacctgtgtggggaatgacagtttcataggactcagttctgtgcgtttgctttctttgtatgataatcaaattactacagttgcaccaggggcatttgatactctccattctttatctactctaaacctcttggccaatccttttaactgtaactgctacctggcttggttgggagagtggctgagaaagaagagaattgtcacgggaaatcctagatgtcaaaaaccatacttcctgaaagaaatacccatccaggatgtggccattcaggacttcacttgtgatgacggaaatgatgacaatagttgctccccactttctcgctgtcctactgaatgtacttgcttggatacagtcgtccgatgtagcaacaagggtttgaaggtcttgccgaaaggtattccaagagatgtcacagagttgtatctggatggaaaccaatttacactggttcccaaggaactctccaactacaaacatttaacacttatagacttaagtaacaacagaataagcacgctttctaatcagagcttcagcaacatgacccagctcctcaccttaattcttagttacaaccgtctgagatgtattcctcctcgcacctttgatggattaaagtctcttcgattactttctctacatggaaatgacatttctgttgtgcctgaaggtgctttcaatgatctttctgcattatcacatctagcaattggagccaaccctctttactgtgattgtaacatgcagtggttatccgactgggtgaagtcggaatataaggagcctggaattgctcgttgtgctggtcctggagaaatggcagataaacttttactcacaactccctccaaaaaatttacctgtcaaggtcctgtggatgtcaatattctagctaagtgtaacccctgcctatcaaatccgtgtaaaaatgatggcacatgtaatagtgatccagttgacttttaccgatgcacctgtccatatggtttcaaggggcaggactgtgatgtcccaattcatgcctgcatcagtaacccatgtaaacatggaggaacttgccacttaaaggaaggagaagaagatggattctggtgtatttgtgctgatggatttgaaggagaaaattgtgaagtcaacgttgatgattgtgaagataatgactgtgaaaataattctacatgtgtcgatggcattaataactacacatgcctttgcccacctgagtatacaggtgagttgtgtgaggagaagctggacttctgtgcccaggacctgaacccctgccagcacgattcaaagtgcatcctaactccaaagggattcaaatgtgactgcacaccagggtacgtaggtgaacactgcgacatcgattttgacgactgccaagacaacaagtgtaaaaacggagcccactgcacagatgcagtgaacggctatacgtgcatatgccccgaaggttacagtggcttgttctgtgagttttctccacccatggtcctccctcgtagcgctcatcaccatcatcatcactga

SEQ ID NO: 16 - protein, 1129 aa, Homo sapiens
MRGVGWQMLSLSLGLVLAILNKVAPQACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSGHQSFMAPSCSVLHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSAKEQYFIPGTEDYRSKLSGDCFADLACPEKCRCEGTTVDCSNQKLNKIPEHIPQYTAELRLNNNEFTVLEATGIFKKLPQLRKINFSNNKITDIEEGAFEGASGVNEILLTSNRLENVQHKMFKGLESLKTLMLRSNRITCVGNDSFIGLSSVRLLSLYDNQITTVAPGAFDTLHSLSTLNLLANPFNCNCYLAWLGEWLRKKRIVTGNPRCQKPYFLKEIPIQDVAIQDFTCDDGNDDNSCSPLSRCPTECTCLDTVVRCSNKGLKVLPKGIPRDVTELYLDGNQFTLVPKELSNYKHLTLIDLSNNRISTLSNQSFSNMTQLLTLILSYNRLRCIPPRTFDGLKSLRLLSLHGNDISVVPEGAFNDLSALSHLAIGANPLYCDCNMQWLSDWVKSEYKEPGIARCAGPGEMADKLLLTTPSKKFTCQGPVDVNILAKCNPCLSNPCKNDGTCNSDPVDFYRCTCPYGFKGQDCDVPIHACISNPCKHGGTCHLKEGEEDGFWCICADGFEGENCEVNVDDCEDNDCENNSTCVDGINNYTCLCPPEYTGELCEEKLDFCAQDLNPCQHDSKCILTPKGFKCDCTPGYVGEHCDIDFDDCQDNKCKNGAHCTDAVNGYTCICPEGYSGLFCEFSPPMVLPRSAHHHHHH

SEQ ID NO: 17 - DNA, 717 bp, Mus musculus
  CDS (1)..(717)
atgtacaggatgcaactcctgtcttgcattgcactaagtcttgcacttgtcacgaattcattgcactgccctgccgcctgtacctgtagcaacaatatcgtagactgtcgtgggaaaggtctcactgagatccccacaaatcttccagagaccatcacagaaatacgtttggaacagaacagcatcagagtcatccctcctggagctttctcaccatataaaaagcttagacgactggacctgagcaataatcagatctctgaacttgcaccagatgctttccaaggactacgctctctgaattcacttgtcctctatggaaataaaatcacagaactccccaaaagtttatttgaaggactgttttccttacagctcctattattgaatgccaacaagataaactgccttcgggtagatgcttttcaggatctccacaacttgaaccttctctccctatatgacaacaagcttcagaccgtcgccaaggggaccttttcagctcttcgggccattcaaactatgcatttggcccagaacccctttatttgtgactgccatctcaagtggctagcggattatctccataccaacccgattgagaccagtggtgcccgttgcaccagcccccgccgcctggcaaacaaaagaattggacagatcaaaagcaagaaattccgttgttcaggtagcgctcatcaccatcatcatcactga

SEQ ID NO: 18 - protein, 238 aa, Mus musculus
MYRMQLLSCIALSLALVTNSLHCPAACTCSNNIVDCRGKGLTEIPTNLPETITEIRLEQNSIRVIPPGAFSPYKKLRRLDLSNNQISELAPDAFQGLRSLNSLVLYGNKITELPKSLFEGLFSLQLLLLNANKINCLRVDAFQDLHNLNLLSLYDNKLQTVAKGTFSALRAIQTMHLAQNPFICDCHLKWLADYLHTNPIETSGARCTSPRRLANKRIGQIKSKKFRCSGSAHHHHHH

SEQ ID NO: 19 - DNA, 726 bp, Homo sapiens
  CDS (1)..(726)
atgtacaggatgcaactcctgtcttgcattgcactaagtcttgcacttgtcacgaattcactgtcctccggctcctgcccggccatgtgcacctgcagcaatggcatcgtggactgtcgtggaaaaggcctcactgccatcccggccaacctgcccgagaccatgacggagatacgcctggagctgaacggcatcaagtccatccctcctggagccttctcaccctacagaaagctacggaggatagacctgagcaacaatcagatcgctgagattgcacccgacgccttccagggcctccgctccctgaactcgctggtcctctatggaaacaagatcacagacctcccccgtggtgtgtttggaggcctatacaccctacagctcctgctcctgaatgccaacaagatcaactgcatccggcccgatgccttccaggacctgcagaacctctcactgctctccctgtatgacaacaagatccagagcctcgccaagggcactttcacctccctgcgggccatccagactctgcacctggcccagaaccctttcatttgcgactgtaacctcaagtggctggcagacttcctgcgcaccaatcccatcgagacgagtggtgcccgctgtgccagtccccggcgcctcgccaacaagcgcatcgggcagatcaagagcaagaagttccggtgctcaggtagcgctcatcaccatcatcatcactga

SEQ ID NO: 20 - protein, 241 aa, Homo sapiens
MYRMQLLSCIALSLALVTNSLSSGSCPAMCTCSNGIVDCRGKGLTAIPANLPETMTEIRLELNGIKSIPPGAFSPYRKLRRIDLSNNQIAEIAPDAFQGLRSLNSLVLYGNKITDLPRGVFGGLYTLQLLLLNANKINCIRPDAFQDLQNLSLLSLYDNKIQSLAKGTFTSLRAIQTLHLAQNPFICDCNLKWLADFLRTNPIETSGARCASPRRLANKRIGQIKSKKFRCSGSAHHHHHH

SEQ ID NO: 21 - DNA, 726 bp, Homo sapiens
  CDS (1)..(726)
atgtacaggatgcaactcctgtcttgcattgcactaagtcttgcacttgtcacgaattcagccaactccatctcctgcccttcgccctgcacgtgcagcaataacatcgtggactgtcgaggaaagggcttgatggagattcctgccaacttgccggagggcatcgtcgaaatacgcctagaacagaactccatcaaagccatccctgcaggagccttcacccagtacaagaaactgaagcgaatagacatcagcaagaatcagatatcggatattgctccagatgccttccagggcctgaaatcactcacatcgctggtcctgtatgggaacaagatcaccgagattgccaagggactgtttgatgggctggtgtccctacagctgctcctcctcaatgccaacaagatcaactgcctgcgggtgaacacgtttcaggacctgcagaacctcaacttgctctccctgtatgacaacaagctgcagaccatcagcaaggggctcttcgcccctctgcagtccatccagacactccacttagcccaaaacccatttgtgtgcgactgccacttgaagtggctggccgactacctccaggacaaccccatcgagacaagcggggcccgctgcagcagcccgcgccgactcgccaacaagcgcatcagccagatcaagagcaagaagttccggtgctcaggtagcgctcatcaccatcatcatcactga

SEQ ID NO: 22 - protein, 241 aa, Homo sapiens
MYRMQLLSCIALSLALVTNSANSISCPSPCTCSNNIVDCRGKGLMEIPANLPEGIVEIRLEQNSIKAIPAGAFTQYKKLKRIDISKNQISDIAPDAFQGLKSLTSLVLYGNKITEIAKGLFDGLVSLQLLLLNANKINCLRVNTFQDLQNLNLLSLYDNKLQTISKGLFAPLQSIQTLHLAQNPFVCDCHLKWLADYLQDNPIETSGARCSSPRRLANKRISQIKSKKFRCSGSAHHHHHH

SEQ ID NO: 23 - DNA, 1362 bp, Homo sapiens
  CDS (1)..(1362)
atgtacaggatgcaactcctgtcttgcattgcactaagtcttgcacttgtcacgaattcacttcgtcaggaagattttccacctcgcattgttgaacacccttcagacctgattgtctcaaaaggagaacctgcaactttgaactgcaaagctgaaggccgccccacacccactattgaatggtacaaagggggagagagagtggagacagacaaagatgaccctcgctcacaccgaatgttgctgccgagtggatctttatttttcttacgtatagtacatggacggaaaagtagacctgatgaaggagtctatgtctgtgtagcaaggaattaccttggagaggctgtgagccacaatgcatcgctggaagtagccatacttcgggatgacttcagacaaaacccttcggatgtcatggttgcagtaggagagcctgcagtaatggaatgccaacctccacgaggccatcctgagcccaccatttcatggaagaaagatggctctccactggatgataaagatgaaagaataactatacgaggaggaaagctcatgatcacttacacccgtaaaagtgacgctggcaaatatgtttgtgttggtaccaatatggttggggaacgtgagagtgaagtagccgagctgactgtcttagagagaccatcatttgtggagtccaagtacggccctccttgccctccctgccctgcccctgagttcgagggcggacctagcgtgttcctgttccctcctaagcctaaggacaccctgatgatctcccggacccctgaggtgacctgtgtggtggtggacgtgtcccaggaggaccctgaggtccagttcaactggtacgtggacggcgtggaggtgcacaacgccaagaccaagcctcgggaggagcagttcaattccacctaccgggtggtgtctgtgctgaccgtgctgcaccaggactggctgaacggcaaagaatacaagtgtaaggtctccaacaagggcctgccctcctccatcgagaaaaccatctccaaggccaagggccagcctagggagcctcaggtgtacaccctgcctcctagccaggaagagatgaccaagaaccaggtgtccctgacctgtctggtgaagggcttctacccttccgacatcgccgtggagtgggagtccaacggccagcctgagaacaactacaagaccacccctcctgtgctggactccgacggctccttcttcctgtactccaggctgaccgtggacaagtcccggtggcaggagggcaacgtcttttcctgctccgtgatgcacgaggccctgcacaaccactacacccagaagtccctgtccctgtctctgggctga

SEQ ID NO: 24 - protein, 453 aa, Homo sapiens
MYRMQLLSCIALSLALVTNSLRQEDFPPRIVEHPSDLIVSKGEPATLNCKAEGRPTPTIEWYKGGERVETDKDDPRSHRMLLPSGSLFFLRIVHGRKSRPDEGVYVCVARNYLGEAVSHNASLEVAILRDDFRQNPSDVMVAVGEPAVMECQPPRGHPEPTISWKKDGSPLDDKDERITIRGGKLMITYTRKSDAGKYVCVGTNMVGERESEVAELTVLERPSFVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG